(19) United States
(12) Patent Application Publication
(10) Pub. No.: US 2022/0280289 A1
(43) Pub. Date: Sep. 8, 2022

(54) PROSTHETIC HEART VALVES WITH COMMISSURE POST PROTRUSIONS FOR COMMISSURE ASSEMBLY POSITION LOCKING

(71) Applicant: Edwards Lifesciences Corporation, Irvine, CA (US)

(72) Inventors: Noam Nir, Pardes-Hanna (IL); Danny M. Garmahi, Hadera (IL)

(73) Assignee: Edwards Lifesciences Corporation, Irvine, CA (US)

(21) Appl. No.: 17/826,029

(22) Filed: May 26, 2022

Related U.S. Application Data
(63) Continuation of application No. PCT/US2020/062225, filed on Nov. 25, 2020.
(60) Provisional application No. 62/945,029, filed on Dec. 6, 2019.

(51) Int. Cl. A61F 2/24

(52) U.S. Cl. CPC A61F 2/2418; A61F 2/2436; A61F 2/2439; A61F 2250/001

(57) ABSTRACT

A prosthetic heart valve includes an expandable annular frame and a valvular structure. The annular frame has an inflow end and an outflow end separated from the inflow end along an axial direction of the frame. The annular frame can also comprise a plurality of commissure support posts. The valvular structure is supported within the annular frame and includes a plurality of leaflets. Each leaflet has a pair of tabs. The tabs of adjacent leaflets are paired together to form a commissure tab assembly. Each commissure tab assembly is coupled to a corresponding one of the commissure support posts. Each commissure support post has a first portion with one or more protrusions arranged along an axial direction of the frame so as to restrain axial motion of the respective commissure tab assembly.

### CROSS REFERENCE TO RELATED APPLICATIONS

This application is a continuation of a PCT Patent Application No. PCT/US2020/062225, entitled “PROSTHETIC HEART VALVES WITH COMMISSURE POST PROTRUSIONS FOR COMMISSURE ASSEMBLY POSITION LOCKING,” filed Nov. 25, 2020, which claims the benefit of U.S. Provisional Application No. 62/945,029, entitled “PROSTHETIC HEART VALVE ASSEMBLIES AND METHODS,” filed Dec. 6, 2019, all of which are incorporated by reference herein in their entirety.

### FIELD

The present disclosure relates to prosthetic heart valves, in particular, to attachment of commissure assemblies of a valvular structure of a prosthetic heart valve to commissure posts of a frame of the prosthetic heart valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery device and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery device so that the prosthetic valve can self-expand to its functional size.

Prosthetic valves that rely on a mechanical actuator for expansion can be referred to as “mechanically expandable” prosthetic heart valves. The actuator typically takes the form of pull cables, sutures, wires and/or shafts that are configured to transmit expansion forces from a handle of the delivery apparatus to the prosthetic valve.

Expandable, transcatheter heart valves can comprise a cylindrical metal frame or stent and prosthetic leaflets mounted inside the frame. The leaflets can be attached to commissure posts of the frame via commissure tab assemblies. Each commissure tab assembly can be preassembled by connecting tabs of adjacent leaflets to each other. The outermost portion of the commissure assembly can be wrapped around the commissure post, and the commissure assembly can then be attached to a respective commissure post of the frame by a suture.

Such commissure mounting methods can present fabrication challenges, as accurate positioning of the commissure assembly over the commissure post can be difficult and the position of the commissure assembly on the commissure post may change during assembly handling, crimping, or valve expansion. Undesirable disposition of the commissure assembly relative to the commissure post can include rotation of the commissure assembly over the commissure post such that the leaflet arrangement is no longer centered within the frame, axial sliding of the commissure assembly along the commissure post such that a part of the commissure assembly slides off an edge of the commissure post (which may occur during valve crimping), and/or axial sliding of the commissure assembly along the commissure post such that a part of the commissure assembly is compressed against a frame junction (which may also occur during valve crimping).

Accordingly, a need exists for improved prosthetic heart valves and methods for securing leaflet assemblies to a frame of the prosthetic heart valve.

### SUMMARY

Described herein are embodiments of prosthetic heart valves and methods for assembling prosthetic heart valves. In some embodiments, an expandable annular frame for a prosthetic heart valve may comprise a plurality of commissure support posts. Each commissure support post has a protrusion that extends in a direction perpendicular to a longitudinal axis of the annular frame and that restrains movement along the commissure support post of a commissure tab assembly of leaflets of a valvular structure. In some embodiments, the protrusion extends radially inward or along a circumferential direction of the annular frame. In some embodiments, sutures can be passed through one or more apertures of the protrusion, or through open slots formed by the protrusion, to further secure the commissure tab assembly to the commissure support post. In some embodiments, each commissure support post includes protrusions that restrain movement of the commissure tab assembly at opposite end portions of the commissure tab assembly.

In one representative embodiment, a prosthetic heart valve can include an expandable annular frame and a valvular structure. The annular frame can have a longitudinal axis and can comprise a plurality of commissure support posts. The valvular structure can be supported by the frame and can comprise a plurality of leaflets. Each leaflet can have a pair of tabs. The tabs of adjacent leaflets can be paired together to form a commissure tab assembly. There can be one commissure post for each commissure tab assembly. Each commissure tab assembly can be coupled to the respective commissure support post with one tab wrapped at least partially around the commissure support post in a first direction and the other tab wrapped at least partially around the commissure support post in a second direction. At least a first portion of each commissure support post can include at least one first protrusion that extends in a direction perpendicular to the longitudinal axis and is constructed to restrain axial motion of the respective commissure tab assembly in a downstream direction.

In another representative embodiment, a method of assembling a prosthetic heart valve, can comprise disposing commissure tab assemblies on respective commissure support posts of an expandable annular frame having a longitudinal axis. Each commissure support post can have a first portion and a second portion. The first portion can be closer to an axial end of the annular frame than the second portion. The method can further comprise coupling each commissure tab assembly to the respective commissure support post. The disposing can include wrapping one leaflet tab of the commissure tab assembly at least partially around the respective commissure support post in a first direction, and wrapping another leaflet tab of the commissure tab assembly at least partially around the respective commissure support post in a second direction. At least the first portion of each commissure support post can include at least one first protrusion that extends in a direction perpendicular to the longitudinal axis and is constructed to restrain movement of the respective commissure tab assembly toward said axial end of the annular frame.

In another representative embodiment, a prosthetic heart valve comprises an annular frame and a valvular structure. The annular frame can be radially collapsible and expandable between a radially-compressed configuration and a radially-expanded configuration. The annular frame can have an inflow end and an outflow end separated from the inflow end along an axial direction of the frame. The annular frame can comprise a plurality of commissure support posts. The valvular structure can be supported within the annular frame and can comprise a plurality of leaflets. Each leaflet can have a pair of tabs. The tabs of adjacent leaflets can be paired together to form a commissure tab assembly. There can be one commissure support post for each commissure tab assembly. Each commissure tab assembly can be coupled to the corresponding commissure support post with one tab wrapped at least partially around the commissure support post in a first direction and the other tab wrapped at least partially around the commissure support post in a second direction. Each commissure support post can have a first portion with a pair of first protrusions that project in opposite directions along a circumferential direction of the annular frame. For each commissure support post, the corresponding commissure tab assembly can be disposed along the axial direction between the first portion and the inflow end of the annular frame. For each commissure support post, the pair of first protrusions can be arranged along the axial direction so as to restrain axial motion of the respective commissure tab assembly in a downstream direction.

In another representative embodiment, a prosthetic heart valve comprises an annular frame and a valvular structure. The annular frame can be radially collapsible and expandable between a radially-compressed configuration and a radially-expanded configuration. The annular frame can have an inflow end and an outflow end separated from the inflow end along an axial direction of the frame. The annular frame can comprise a plurality of commissure support posts. The valvular structure can be supported within the annular frame and can comprise a plurality of leaflets. Each leaflet can have a pair of tabs. The tabs of adjacent leaflets can be paired together to form a commissure tab assembly. There can be one commissure support post for each commissure tab assembly. Each commissure tab assembly can be coupled to the corresponding commissure support post with one tab wrapped at least partially around the commissure support post in a first direction and the other tab wrapped at least partially around the commissure support post in a second direction. Each commissure support post can have a first portion with a first protrusion that projects along a radial direction of the annular frame toward an interior of the annular frame. For each commissure support post, the corresponding commissure tab assembly can be disposed along the axial direction between the first portion and the inflow end of the annular frame. For each commissure support post, the first protrusion can be arranged along the axial direction so as to restrain axial motion of the respective commissure tab assembly in a downstream direction.

In another representative embodiment, a prosthetic heart valve comprises an annular frame and a valvular structure. The annular frame can be radially collapsible and expandable between a radially-compressed configuration and a radially-expanded configuration. The annular frame can have an inflow end and an outflow end separated from the inflow end along an axial direction of the frame. The annular frame can comprise a plurality of commissure support posts. The valvular structure can be supported within the annular frame and can comprise a plurality of leaflets. Each leaflet can have a pair of tabs. The tabs of adjacent leaflets can be paired together to form a commissure tab assembly. There is one commissure support post for each commissure tab assembly. Each commissure tab assembly can be coupled to the corresponding commissure support post with one tab wrapped at least partially around the commissure support post in a first direction and the other tab wrapped at least partially around the commissure support post in a second direction. Each commissure support post can have a first portion with a pair of first protrusions that project in opposite directions along a circumferential direction of the annular frame. Each first protrusion can have a neck portion with a first width and an end portion with a second width greater than the first width. The neck portion can be disposed along the circumferential direction between the end portion and a central portion of the first portion of the respective commissure support post such that at least one slot is formed between a surface of the central portion and a facing surface of the end portion. For each commissure support post, the corresponding commissure tab assembly can be disposed along the axial direction between the first portion and the inflow end of the annular frame. For each commissure support post, the pair of first protrusions can be arranged along the axial direction so as to restrain axial motion of the respective commissure tab assembly in a downstream direction.

In another representative embodiment, a prosthetic heart valve comprises an annular frame and a valvular structure. The annular frame can be radially collapsible and expandable between a radially-compressed configuration and a radially-expanded configuration. The annular frame can have an inflow end and an outflow end separated from the inflow end along an axial direction of the frame. The annular frame can comprise a plurality of commissure support posts. The valvular structure can be supported within the annular frame and can comprise a plurality of leaflets. Each leaflet can have a pair of tabs. The tabs of adjacent leaflets can be paired together to form a commissure tab assembly. There can be one commissure support post for each commissure tab assembly. Each commissure tab assembly can be coupled to the corresponding commissure support post with one tab wrapped at least partially around the commissure support post in a first direction and the other tab wrapped at least partially around the commissure support post in a second direction. Each commissure support post can have a first portion with a first protrusion that projects along a radial direction of the annular frame toward an interior of the annular frame. Each first protrusion can have a neck portion with a first width and an end portion with a second width greater than the first width. The neck portion can be disposed along the radial direction between the end portion and a central portion of the first portion of the respective commissure support post such that at least one slot is formed between a surface of the central portion and a facing surface of the end portion. For each commissure support post, the corresponding commissure tab assembly can be disposed along the axial direction between the first portion and the inflow end of the annular frame. For each commissure support post, the first protrusion can be arranged along the axial direction so as to restrain axial motion of the respective commissure tab assembly in a downstream direction.

In another representative embodiment, a prosthetic heart valve comprises an annular frame and a valvular structure. The annular frame can be radially collapsible and expandable between a radially-compressed configuration and a radially-expanded configuration. The annular frame can have an inflow end and an outflow end separated from the inflow end along an axial direction of the frame. The annular frame can comprise a plurality of commissure support posts. The valvular structure can be supported within the annular frame and can comprise a plurality of leaflets. Each leaflet can have a pair of tabs. The tabs of adjacent leaflets can be paired together to form a commissure tab assembly. Each commissure tab assembly can be coupled to a respective commissure support post. The annular frame can comprise means for restraining axial motion of the commissure tab assemblies.

In another representative embodiment, a method of assembling a prosthetic heart valve can comprise disposing commissure tab assemblies of leaflets of a valvular structure on respective commissure support posts of an annular frame. The annular frame can be radially collapsible and expandable between a radially-compressed configuration and a radially-expanded configuration. The annular frame can have an inflow end and an outflow end separated from the inflow end along an axial direction of the frame. The annular frame can comprise a plurality of the commissure support posts. There can be one commissure support post for each commissure tab assembly. The valvular structure can comprise a plurality of leaflets. Each leaflet can have a pair of tabs. Each commissure tab assembly can be formed by paired tabs of adjacent leaflets. The method can further comprise coupling each commissure tab assembly to its respective commissure support post. Each commissure support post can have a first portion with a pair of first protrusions that project in opposite directions along a circumferential direction of the annular frame. For each commissure support post, the corresponding commissure tab assembly can be disposed along the axial direction between the first portion and the inflow end of the annular frame. For each commissure support post, the pair of first protrusions can be arranged along the axial direction so as to restrain axial motion of the respective commissure tab assembly in a downstream direction.

In another representative embodiment, a method of assembling a prosthetic heart valve can comprise disposing commissure tab assemblies of leaflets of a valvular structure on respective commissure support posts of an annular frame. The annular frame can be radially collapsible and expandable between a radially-compressed configuration and a radially-expanded configuration. The annular frame can have an inflow end and an outflow end separated from the inflow end along an axial direction of the frame. The annular frame can comprise a plurality of the commissure support posts. There can be one commissure support post for each commissure tab assembly. The valvular structure can comprise a plurality of leaflets. Each leaflet can have a pair of tabs. Each commissure tab assembly can be formed by paired tabs of adjacent leaflets. The method can further comprise coupling each commissure tab assembly to its respective commissure support post. Each commissure support post can have a first portion with a first protrusion that projects along a radial direction of the annular frame toward an interior of the annular frame. For each commissure support post, the corresponding commissure tab assembly can be disposed along the axial direction between the first portion and the inflow end of the annular frame. For each commissure support post, the first protrusion can be arranged along the axial direction so as to restrain axial motion of the respective commissure tab assembly in a downstream direction.

In another representative embodiment, a method of assembling a prosthetic heart valve can comprise disposing commissure tab assemblies of leaflets of a valvular structure on respective commissure support posts of an annular frame. The annular frame can be radially collapsible and expandable between a radially-compressed configuration and a radially-expanded configuration. The annular frame can have an inflow end and an outflow end separated from the inflow end along an axial direction of the frame. The annular frame can comprise a plurality of the commissure support posts. There can be one commissure support post for each commissure tab assembly. The valvular structure can comprise a plurality of leaflets. Each leaflet can have a pair of tabs. Each commissure tab assembly can be formed by paired tabs of adjacent leaflets. The method can further comprise coupling each commissure tab assembly to its respective commissure support post. Each commissure support post can have a first portion with a pair of first protrusions that project in opposite directions along a circumferential direction of the annular frame. Each first protrusion can have a neck portion with a first width and an end portion with a second width greater than the first width. The neck portion can be disposed along the circumferential direction between the end portion and a central portion of the first portion of the respective commissure support post such that at least one slot is formed between a surface of the central portion and a facing surface of the end portion. For each commissure support post, the corresponding commissure tab assembly can be disposed along the axial direction between the first portion and the inflow end of the annular frame. For each commissure support post, the pair of first protrusions can be arranged along the axial direction so as to restrain axial motion of the respective commissure tab assembly in a downstream direction.

In another representative embodiment, a method of assembling a prosthetic heart valve can comprise disposing commissure tab assemblies of leaflets of a valvular structure on respective commissure support posts of an annular frame. The annular frame can be radially collapsible and expandable between a radially-compressed configuration and a radially-expanded configuration. The annular frame can have an inflow end and an outflow end separated from the inflow end along an axial direction of the frame. The annular frame can comprise a plurality of the commissure support posts. There can be one commissure support post for each commissure tab assembly. The valvular structure can comprise a plurality of leaflets. Each leaflet can have a pair of tabs. Each commissure tab assembly can be formed by paired tabs of adjacent leaflets. The method can further comprise coupling each commissure tab assembly to its respective commissure support post. Each commissure support post can have a first portion with a first protrusion that projects along a radial direction of the annular frame toward an interior of the annular frame. Each first protrusion can have a neck portion with a first width and an end portion with a second width greater than the first width. The neck portion can be disposed along the radial direction between the end portion and a central portion of the first portion of the respective commissure support post such that at least one slot is formed between a surface of the central portion and a facing surface of the end portion. For each commissure support post, the corresponding commissure tab assembly can be disposed along the axial direction between the first portion and the inflow end of the annular frame. For each commissure support post, the first protrusion can be arranged along the axial direction so as to restrain axial motion of the respective commissure tab assembly in a downstream direction.

In another representative embodiment, a method of assembling a prosthetic heart valve can comprise disposing commissure tab assemblies of leaflets of a valvular structure on respective commissure support posts of an annular frame. The annular frame can be radially collapsible and expandable between a radially-compressed configuration and a radially-expanded configuration. The annular frame can have an inflow end and an outflow end separated from the inflow end along an axial direction of the frame. The annular frame can comprise a plurality of the commissure support posts. The valvular structure can comprise a plurality of leaflets. Each leaflet can have a pair of tabs. Each commissure tab assembly can be formed by paired tabs of adjacent leaflets. The method can further comprise coupling each commissure tab assembly to its respective commissure support post. The annular frame can comprise means for restraining axial motion of the commissure tab assemblies.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE DRAWINGS

FIG. 1 is a perspective view of an exemplary embodiment of a prosthetic heart valve.

FIG. 2 is a plan view of an individual leaflet used to form a leaflet assembly for a prosthetic heart valve.

FIGS. 3A-3B are schematic illustrations of various stages of assembling a commissure tab assembly to a commissure support post as viewed along the longitudinal axis of the prosthetic heart valve.

FIG. 3C is a schematic illustration of the commissure tab assembly secured to the commissure support post as viewed along the radial direction from an exterior of the prosthetic heart valve.

FIGS. 4A-4B show perspective views of a first example of a commissure support post without and with a commissure tab assembly installed, respectively.

FIGS. 5A-5B show perspective views of a second example of a commissure support post without and with a commissure tab assembly installed, respectively.

FIGS. 6A-6B show perspective views of a third example of a commissure support post without and with a commissure tab assembly installed, respectively.

FIGS. 7A-7B show perspective views of a fourth example of a commissure support post without and with a commissure tab assembly installed, respectively.

FIGS. 8A-8B show perspective views of a fifth example of a commissure support post without and with a commissure tab assembly installed, respectively.

FIGS. 9A-9B show perspective views of a sixth example of a commissure support post without and with a commissure tab assembly installed, respectively.

FIGS. 10A-10B show perspective views of a seventh example of a commissure support post without and with a commissure tab assembly installed, respectively.

FIGS. 11A-11B show perspective views of an eighth example of a commissure support post without and with a commissure tab assembly installed, respectively.

FIG. 12A shows an exemplary prosthetic valve delivery apparatus that can be used for implanting the prosthetic heart valve of FIG. 1.

FIG. 12B shows a perspective view of the distal end portion of the delivery apparatus and the prosthetic valve shown in FIG. 1, showing the prosthetic valve in a radially-expanded state.

### DETAILED DESCRIPTION

Described herein are examples of prosthetic heart valves, annular frames with commissure support posts and leaflet assemblies for prosthetic heart valves, and methods for assembling leaflet assemblies to commissure support posts of annular frames to form prosthetic heart valves. The prosthetic heart valve may include an annular frame with commissure support posts and a valvular structure formed by a leaflet assembly supported by the frame. The leaflet assembly may include a plurality of leaflets, each of which has a pair of tabs. The tabs of adjacent leaflets can be coupled together to form a commissure tab assembly, and the leaflet assembly can be supported by the frame via the commissure tab assemblies coupled to the commissure support posts, respectively. The commissure support posts can have one or more protrusions, which extend either radially inward or circumferentially. These protrusions can restrain movement of the respective commissure tab assembly along its commissure support post. In some embodiments, the commissure tab assembly can be further coupled to the commissure support post via one or more sutures extending through apertures in or slots formed by the protrusions. As a result, a position of the leaflet assembly for a prosthetic heart valve may be effectively locked in place during assembly and use of the prosthetic heart valve, and a time and effort for securing the leaflet assembly to the frame of the prosthetic heart valve may be reduced.

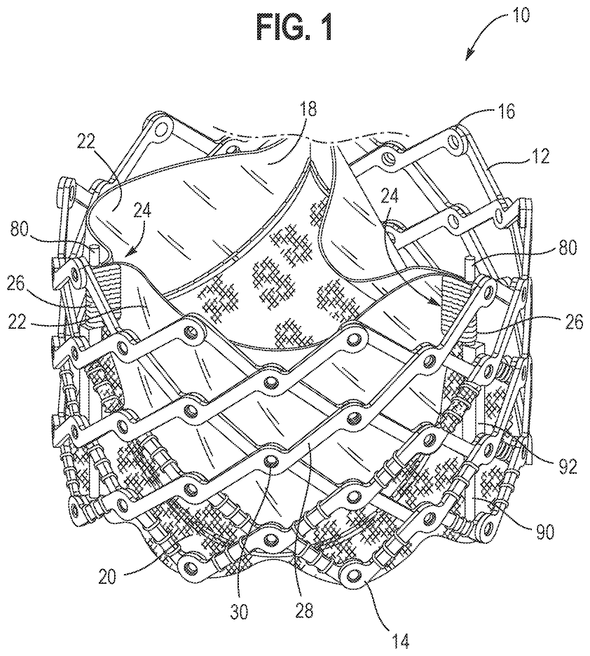

FIG. 1 shows an exemplary prosthetic heart valve 10, according to one or more embodiments of the disclosed subject matter. The prosthetic heart valve 10 can be radially compressible and expandable between a radially-compressed configuration for delivery into a patient and a radially-expanded configuration (as shown in FIG. 1). In particular embodiments, the prosthetic heart valve 10 can be implanted within the native aortic annulus, although it can also be implanted at other locations in the heart, including within the native mitral valve, the native pulmonary valve, or the native tricuspid valve.

The prosthetic heart valve 10 can include an annular stent or frame 12 having a first axial end 14 and a second axial end 16. In the depicted embodiment, the first axial end 14 can be an inflow end, and the second axial end 16 can be an outflow end. The outflow end 16 can be coupled to a delivery apparatus for delivering and implanting the prosthetic heart valve within the native aortic valve is a transfemoral, retrograde delivery approach. Thus, in the delivery configuration of the prosthetic heart valve, the outflow end 16 is the proximal-most end of the prosthetic valve. In other embodiments, the inflow end 14 can be coupled to the delivery apparatus, depending on the particular native valve being replaced and the delivery technique that is used (e.g., trans-septal, transapical, etc.). For example, the inflow end 14 can be coupled to the delivery apparatus (and therefore would be the proximal-most end of the prosthetic heart valve in the delivery configuration) when delivering the prosthetic heart valve to the native mitral valve via a trans-septal delivery approach.

As shown in FIG. 1, frame 12 can include a plurality of interconnected struts 28 arranged in a lattice-type pattern (e.g., an array of angled struts). The struts 28 are shown as positioned diagonally, or offset at an angle relative to, and radially offset from, a longitudinal axis of the prosthetic heart valve 10 when the prosthetic heart valve 10 is in the expanded configuration. In other implementations, the struts 28 can be offset by a different amount than depicted in FIG. 1, or some or all of the struts 28 can be positioned parallel to the longitudinal axis of the prosthetic heart valve 10.

In the embodiment illustrated in FIG. 1, the struts 28 are pivotably coupled to one another at one or more pivot joints along the length of each strut. For example, each of the struts 28 can be formed with apertures at opposing ends of the strut and apertures spaced along the length of the strut. Respective hinges can be formed at the locations where struts 28 overlap each other via fasteners or pivot members, such as rivets or pins 30 that extend through the apertures. The hinges can allow the struts 28 to pivot relative to one another as the frame 12 is radially expanded or compressed, such as during assembly, preparation, or implantation of the prosthetic heart valve 10. The frame 12, or components thereof (e.g., struts 28 and/or pins 30), can be made of any of various suitable materials, such as stainless steel, a cobalt chromium alloy, or a nickel titanium alloy (“NiTi”), for example Nitinol.

In some embodiments, the frame 12 can be constructed by forming individual components (e.g., the struts and fasteners of the frame) and then mechanically assembling and connecting the individual components together. In other embodiments, the struts 28 are not coupled to each other with respective hinges but are otherwise pivotable or bendable relative to each other to permit radial expansion and contraction of the frame 12. For example, the frame 12 can be formed (e.g., via laser cutting, electroforming or physical vapor deposition) from a single piece of material (e.g., a metal tube). Further details regarding the construction of the frame and the prosthetic heart valve are described in U.S. Pat. Nos. 10,603,165 and 10,806,573, U.S. Patent Application Publication Nos. 2018/0344456 and 2020/0188099, U.S. patent application Ser. No. 17/005,058, and International Publication No. WO-2020/081893, all of which are incorporated herein by reference.

The prosthetic heart valve 10 can further include a plurality of actuators 80 mounted to and spaced around an inner surface of the frame 12. The actuators can be configured to apply corresponding expansion and compression forces to the frame in order to radially expand or compress the prosthetic valve. For example, in the illustrated embodiment, the actuators 80 are linear actuators, each of which comprises an inner member 90 (or piston) and an outer member 92 (or cylinder). The inner member 90 is pivotably coupled to a junction of the frame, such as at the first axial end 14, while the outer member 92 is pivotably coupled to another junction of the frame closer to the second axial end 16. Moving the inner member 90 proximally relative to the outer member 92 and/or moving the outer member 92 distally relative to the inner member 90 can be effective to radially expand the prosthetic valve. Conversely, moving the inner member 90 distally relative to the outer member 92 and/or moving the outer member 92 proximally relative to the inner member 90 can be effective to radially compress the prosthetic valve. The actuators 80 can include locking mechanisms that are configured to retain the prosthetic valve in an expanded state inside the patient's body.

In some embodiments, each of the actuators 80 can be configured to form a releasable connection with one or more respective actuators of a delivery apparatus of a transcatheter delivery system. The actuators of the delivery apparatus can transmit forces from a handle of the delivery apparatus to the actuators 80 for expanding or compressing the prosthetic valve. Further details of the actuators, locking mechanisms and delivery apparatuses for actuating the actuators can be found in U.S. Pat. Nos. 10,603,165 and 10,806,573, and U.S. Patent Application Publication No. 2018/0325665, each of which is incorporated herein by reference in its entirety. Any of the actuators and locking mechanisms disclosed in the previously filed applications can be incorporated in any of the prosthetic valves disclosed herein. Further, any of the delivery apparatuses disclosed in the previously filed applications can be used to deliver and implant any of the prosthetic valves discloses herein.

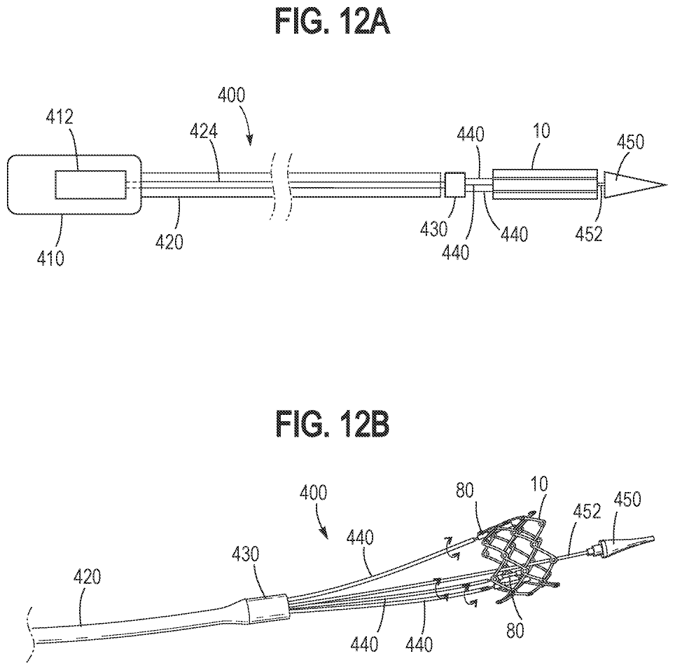

For example, referring to FIGS. 12A-12B, a delivery apparatus 400 that can be used to deliver and implant a prosthetic heart valve, such as prosthetic heart valve 10, in the heart of a patient. The delivery apparatus 400 can have a handle 410, an outer elongated shaft 420 (also referred to herein as a catheter) extending distally from the handle 410, an input torque shaft 424 extending distally from the handle 410 through the outer shaft 420, a gear mechanism 430 (e.g., a gearbox) operatively connected to a distal end portion of the input torque shaft 424, a plurality of output torque shafts 440, and a nose cone 450. The nose cone 450 can be mounted on the distal end portion of an innermost shaft 452 that serves as a guidewire lumen, which can extend coaxially through the input torque shaft 424 and can have a proximal end portion coupled to the handle 410. A guidewire can extend through the guidewire lumen, and a distal end of the delivery apparatus can be advanced over the guidewire to the implant location. Each output torque shaft 440 can have a proximal end portion connected to the gear mechanism 430 and a distal end portion releasably connected to a respective screw of actuator 80. Each output torque shaft 440 can have, for example, a rod, a rigid tube, a cable, a laser cut tube, a hypotube, or any other elongated annular structure (e.g., any tubular or cylindrical structure). A proximal end portion of the input torque shaft 424 can be operatively connected to an actuator, such as a motor 412, housed within or coupled to the handle 410. The motor 412 can be, for example, an electric motor powered by batteries, which can also be housed within the handle 410. Alternatively, the motor 412 can be a hydraulically-driven or a pneumatically-driven motor. The motor 412 can be operable to actuate or rotate the input torque shaft 424, which in turn actuates or rotates the output torque shafts 440 via the gear mechanism 430, which in turn actuates the prosthetic heart valve to radially expand or compress. Further details regarding construction and operation of a delivery apparatus for delivering and implanting a prosthetic heart valve can be found in U.S. Pat. Nos. 9,827,093, 10,076,638, and 10,806,573, all of which are incorporated herein by reference.

In some embodiments, the frame 12, or components thereof (e.g., struts and/or fasteners), can be made of any of various suitable plastically-expandable materials (e.g., stainless steel, etc.) or self-expanding materials (e.g., nickel titanium alloy (NiTi), such as Nitinol), as known in the art. In such configurations, Suitable plastically-expandable materials that can be used to form the frame 12 include, without limitation, stainless steel, biocompatible high-strength alloys (e.g., a cobalt-chromium or a nickel-cobalt-chromium alloys), polymers, or combinations thereof. In particular embodiments, frame 12 is made of a nickel-cobalt-chromium-molybdenum alloy, such as MP35N® alloy (SPS Technologies, Jenkintown, Pa.), which is equivalent to UNS R30035 alloy (covered by ASTM F562-02). MP35N® alloy/UNS R30035 alloy comprises 35% nickel, 35% cobalt, 20% chromium, and 10% molybdenum, by weight. When constructed of a plastically-expandable material, the frame 12 (and thus the prosthetic valve 10) can be crimped to a radially collapsed configuration on a delivery catheter and then expanded inside a patient by an inflatable balloon or equivalent expansion mechanism. Details of exemplary delivery apparatuses that can be used to deliver and implant plastically-expandable prosthetic valves are disclosed in U.S. Pat. Nos. 9,339,384, 10,076,638, and 10,588,744, all of which are incorporated herein by reference. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size. Details of exemplary delivery apparatuses that can be used to deliver and implant self-expandable prosthetic valves are disclosed in U.S. Pat. Nos. 8,652,202 and 9,867,700, both of which are incorporated herein by reference.

In some embodiments, each of the actuators 80 can be used to support a respective commissure 24 (described below). As such, the actuators 80 can include commissure support posts for supporting and securing commissures 24 of a valvular structure 18 to the frame 12. In some embodiments, as shown in FIG. 1, the commissures 24 can be mounted (e.g., sutured) directly to commissure support posts of the actuators 80 of the frame 12 via respective commissure tab assemblies 26. Alternatively or additionally, the frame 12 of the heart valve 10 can include commissure support posts as separate structures from the actuators 80. For example, the commissures 24 can be mounted to commissure support posts that are support struts or posts of the frame 12 that are separate from the actuators 80. In such a configuration, the commissure support posts may be referred to as commissure posts or commissure locking posts. In some embodiments, the commissure support posts (or a subsection thereof to which the commissures 24 are coupled) may be substantially aligned with, or extend along a direction substantially parallel to, the longitudinal axis of the frame 12.

The prosthetic heart valve 10 can also include a valvular structure 18, which is coupled to the frame 12 and configured to regulate the flow of blood through the prosthetic heart valve 10 from the inflow end 14 to the outflow end 16. The valvular structure 18 can include, for example, a leaflet assembly formed by one or more leaflets 22 (three leaflets 22 in the illustrated embodiment) made of a flexible material. In the illustrated embodiment of FIG. 1, the valvular structures is tricuspid structure with three leaflets, with commissures equally spaced around a circumference of the annular frame (e.g., along the circumferential direction of the annular frame). Alternatively, in some embodiments, the valvular structure can be a bicuspid structure with two leaflets, with commissures on diametrically opposite sides of the annular frame from each other.

The leaflets 22 of the leaflet assembly can be made from in whole or part, biological material, bio-compatible synthetic materials, or other such materials. Suitable biological material can include, for example, bovine pericardium (or pericardium from other sources). Further details regarding transcatheter prosthetic heart valves, including the manner in which the valvular structure can be coupled to the frame 12 of the prosthetic heart valve 10, can be found, for example, in U.S. Pat. Nos. 6,730,118, 7,393,360, 7,510,575, 7,993,394, and 8,652,202, and U.S. Patent Application Publication No. 2018/0325665, all of which are incorporated herein by reference in their entireties.

The prosthetic heart valve 10 can also include one or more skirts or sealing members. For example, as shown in FIG. 1, the prosthetic heart valve 10 can include an inner skirt 20 mounted on the inner surface of the frame 12. As shown in FIG. 1, the inner skirt 20 is a circumferential inner skirt that spans an entire circumference of the inner surface of the frame 12. The inner skirt 20 can function as a sealing member to prevent or decrease perivalvular leakage (e.g., when the valve is placed at the implantation site) and as an attachment surface to anchor the leaflets 22 to the frame 12.

The prosthetic heart valve 10 can also include an outer skirt mounted on the outer surface of the frame 12 (not shown in FIG. 1). The outer skirt can function as a sealing member for the prosthetic valve by sealing against the tissue of the native valve annulus and helping to reduce paravalvular leakage past the prosthetic valve. The inner and outer skirts can be formed from any of various suitable biocompatible materials, including any of various synthetic materials (e.g., PET) or natural tissue (e.g., pericardial tissue). The inner and outer skirts can be mounted to the frame using sutures, an adhesive, welding, and/or other means for attaching the skirts to the frame.

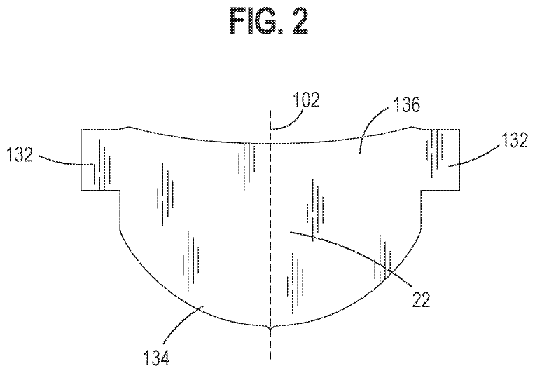

As shown in FIG. 2, each leaflet 22 can comprise a main, cusp edge portion 134, two leaflet tabs (also referred to as commissure tabs) 132 at opposing ends of the cusp edge portion 134, and an upper edge portion 136. The cusp edge portion 134, leaflet tabs 132, and upper edge portion 136 may be arranged around an outer perimeter of the leaflet 22, with the upper edge portion 136 extending between the two leaflet tabs 132 at an upper edge of the leaflet 22 and the cusp edge portion 134 extending between the two leaflet tabs 132 at a lower edge of the leaflet 22. As used here, “upper” and “lower” may be relative to a central longitudinal axis of the prosthetic heart valve 10 when the leaflet assembly is installed and coupled to frame 12 of the prosthetic heart valve 10.

In some embodiments, the cusp edge portion 134 has a curved, scalloped shape (as shown in FIG. 2). Thus, the cusp edge portion 134 may curve between the two leaflet tabs 132. FIG. 2 further illustrates a centerline 102 for each of the individual leaflets 22, which may also be a centerline of the leaflet assembly. For example, when assembled, the centerlines 102 for each of the leaflets 22 may overlap. Further, as shown in FIG. 2, the leaflet tabs 132 may be arranged at opposing ends of the cusp edge portion 134, across the centerline 102 from one another. In some embodiments, the leaflets and/or components of the leaflet assembly may have symmetry with respect to the centerline 102.

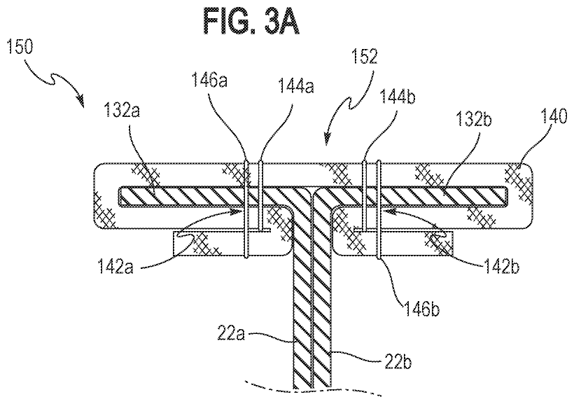

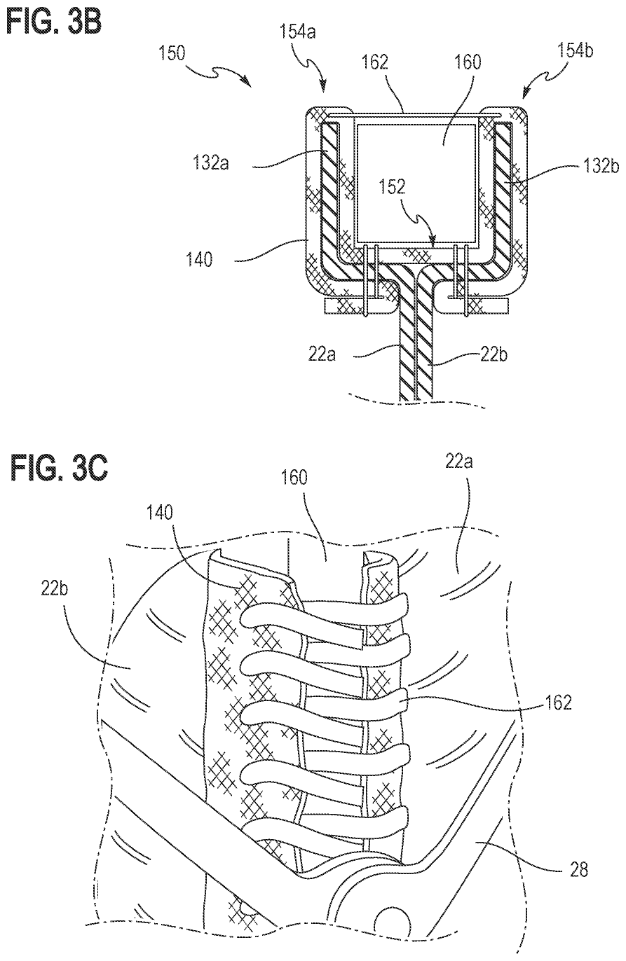

The leaflets 22 can be arranged to form commissures 24 that can be coupled to respective commissure support posts, thereby securing at least a portion of the leaflet assembly to the frame 12. For example, as illustrated in FIG. 3A, a commissure tab assembly 150 can be pre-assembled prior to attachment thereof to the frame 12 by connecting together respective tabs 132a, 132b of a pair of adjacent leaflets 22a, 22b. In some embodiments, the commissure tab assembly 150 can include one or more coupling members 140 coupled to the leaflet tabs 132a, 132b. For example, the coupling member 140 can be a flexible fabric or cloth strip. The coupling member 140 can be configured to protect the leaflets from portions of the frame 12 or actuators 80, and/or to secure tabs 132a, 132b to each other and to the commissure support post 160. As such, the coupling member 140 can extend between the leaflets 22a, 22b and the commissure support post 160, as illustrated in FIGS. 3A-3D. The coupling member 140 and the tabs 132a, 132b can be coupled together in various ways, such as, but not limited to, sutures or adhesive.

In some embodiments, as illustrated in FIG. 3A, the commissure tab assembly 150 can be pre-assembled by disposing leaflet tabs 132a, 132b adjacent to each other. The coupling member 140 can be wrapped around a free end of the leaflet tabs 132a, 132b (i.e., distal from centerline 102 of each leaflet 22). In some embodiments, a portion of the coupling member 140 can be disposed between facing surfaces at the free end of the leaflet tabs 132a, 132b, as shown in FIG. 3B. In some embodiments, reinforcing members can optionally be disposed adjacent to outer surfaces of the leaflet tabs 132a, 132b in a region 142a, 142b, which may be radially adjacent to where the coupling member 140 is disposed between the facing surfaces of the leaflet tabs 132a, 132b (for example, when the commissure tab assembly 150 is wrapped around commissure support post 160) as illustrated in FIG. 3B. The optional reinforcing members can be formed of any material that provides structural support to the commissure tab assembly 150, such as, but not limited to, a thick suture (e.g., Ethibond) or a cloth or fabric strip.

In some embodiments, as illustrated in FIG. 3A, the pre-assembly of the commissure tab assembly 150 can include extending a suture along primary suture line 144a through a left side of the coupling member 140 and left leaflet tab 132a and another suture along primary suture line 144b through a right side of the coupling member 140 and right leaflet tab 132b. Additional sutures can be extended along secondary suture lines to further secure the coupling member 140 to the respective tabs 132a, 132b. For example, as illustrated in FIG. 3A, a suture can be extended along secondary suture line 146a through a folded over portion of the left side of the coupling member 140, left leaflet tab 132a, and a mid-portion of the coupling member 140. Another suture can similarly be extended along secondary suture line 146b through a folded over portion of the right side of the coupling member 140, right leaflet tab 132b, and a mid-portion of the coupling member 140.

In some embodiments, as illustrated in FIGS. 3B-3C, the pre-assembled commissure tab assembly 150 can be attached to a corresponding commissure support post 160. While the leaflet tabs 132a, 132b of the pre-assembled commissure tab assembly may initially extend radially with respect to the frame 12, the tabs 132a, 132b can be flared circumferentially away from each other as illustrated in FIG. 3A in preparation for contacting a central region 152 of the flared commissure tab assembly 150 with a radially-inner surface of the commissure support post 160. Then, as shown in FIGS. 3B-3C, the flared tabs 132a, 132b can be wrapped at least partially around commissure support post 160. For example, one of the tabs 132a can be partially wrapped around the commissure support post 160 in a first direction (e.g., clockwise direction in FIG. 3C) and the other tab 132b can be partially wrapped around the commissure support post 160 in an opposite second direction (e.g., counter-clockwise direction in FIG. 3C).

The commissure tab assembly 150 can then be coupled to the commissure support post 160 to support the leaflet assembly within frame 12. In some embodiments, as illustrated in FIGS. 3B-3C, one or more sutures can be extended along tertiary suture lines 162 to connect together the opposite ends 154a, 154b of the commissure tab assembly 150, e.g., portions of coupling member 140 covering respective ends of the leaflet tabs 132a, 132b. For example, as shown in FIG. 3C, the one or more sutures along tertiary suture lines 162 may form a “shoelace” stitching along a radially outer surface of commissure support post 160, with respective ends of each suture being knotted.

While FIGS. 3A-3C illustrate a specific arrangement and coupling of components for the pre-assembly of the commissure tab assembly 150, other arrangements, coupling, and/or components for the pre-assembly are also possible according to one or more contemplated embodiments. Indeed, folding of coupling member 140, location or existence of reinforcing members, and/or existence, location, or number of suture lines 144a,b, 146a,b, and 162 may be different than illustrated in FIGS. 3A-3C. For example, one or more of suture lines 144a,b, and 146a, b may be omitted in favor of a different form of coupling, such as adhesive bonding.

Moreover, although FIGS. 3B-3C illustrate a specific arrangement and coupling of the commissure tab assembly 150 to commissure support post 160, other arrangements and/or coupling of the commissure tab assembly to the commissure support post 160 are also possible. For example, arrangement of leaflet tabs 132a, 132b, coupling member 140, and/or reinforcing members on the commissure support post 160, cross-sectional shape of commissure support post 160, and/or existence, location, or number of tertiary suture line 162 may be different than illustrated in FIGS. 3B-3C. Further, in some embodiments, the commissures of a leaflet assembly need not be pre-assembled prior to mounting the leaflet assembly on the frame; any of the commissure configurations disclosed herein can be formed when mounting the leaflets to the frame.

In some embodiments, the commissure support post can include one or more protrusions (also referred to as extensions or projections) that are constructed to restrict axial motion of an installed commissure tab assembly along the commissure support post. In some embodiments, the commissure tab assembly may be further secured to the protrusion, for example, by one or more sutures passing through an aperture of or slot formed by the protrusion, to restrict axial motion along the commissure support post and/or rotational motion about the commissure support post.

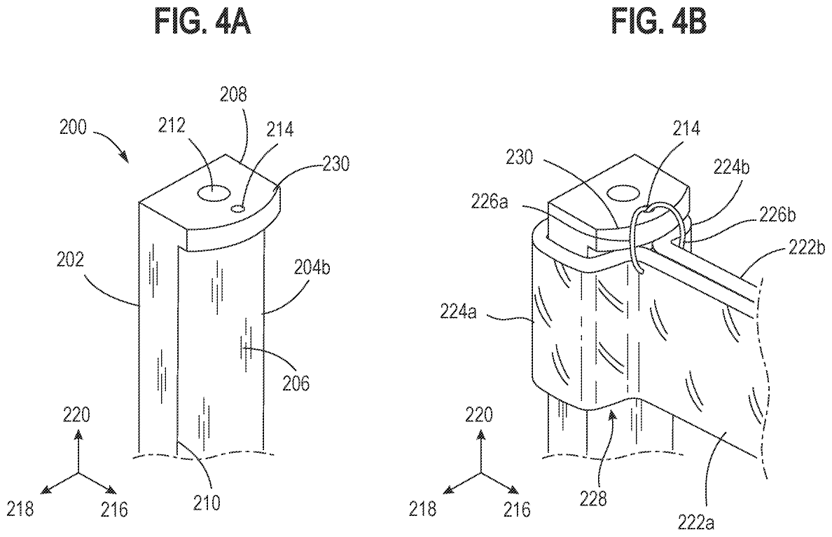

Referring to FIG. 4A, a first example of a commissure support post 200 is illustrated. The commissure support post 200 can have a radially inner side 206, which faces inward toward a centerline of the annular frame, and a radially outer side 202, which faces outward toward an exterior of the annular frame and toward the patient's anatomy when the prosthetic heart valve is implanted. A pair of opposing sides 204a, 204b connects the outer side 202 to the inner side 206 at respective edges thereof. In some embodiments, the commissure support post 200 can have one or more longitudinally-extending internal lumens 212 disposed at a location spaced radially outward from the protrusion 230. For example, when the commissure support post 200 is a part of an actuator 80 of the annular frame 12, internal lumen 212 may be an actuation lumen or locking lumen sized to receive an actuator member (e.g., inner member 90) and/or a locking member.

The commissure support post 200 further includes at least one protrusion 230 at a first end portion 208 thereof. In the illustrated embodiment of FIG. 4A, the at least one protrusion 230 projects inward along radial direction 216 from the inner side 206 of the commissure support post 200, such that at least a portion of the protrusion 230 is closer to a center of the frame 12 than the commissure support post 200. At a second portion 210 remote from the first end portion 208, the commissure support post 200 does not include any protrusions. In some embodiments, the first end portion 208 may be closer to the outflow end 16 of the annular frame 12 than the second portion 210. In other embodiments, the first end portion 208 may be closer to the inflow end 14 of the annular frame 12 than the second portion 210.

Although FIG. 4A illustrates a particular location of protrusion 230 coincident with an end of the commissure support post 200, in some embodiments the protrusion 230 can be located at other positions along the length of the commissure support post 200, such that the commissure support post 200 extends both above and below the protrusion 230 along axial direction 220. In such embodiments, an upper surface of protrusion 230 would not be coincident or coplanar with an upper surface of the commissure support post 200; however, protrusion 230 would be disposed closer to first end portion 208 than the second portion 210.

In the illustrated embodiment of FIGS. 4A-4B, protrusion 230 includes at least one aperture or through-hole 214, through which a suture or other attachment means can be passed. In some embodiments, protrusion 230 is provided with multiple apertures 214, each of which is constructed to receive passage of a respective suture or other attachment means therethrough. Although aperture 214 is shown in FIG. 4A as extending along longitudinal direction 220 (also referred to as the axial direction) with openings at top and bottom surfaces of the protrusion 230, in some embodiments the aperture can instead extend along the circumferential direction 218 with openings at circumferential surfaces of the protrusion 230.

A commissure tab assembly 228 can be coupled to the commissure support post 200 between first end portion 208 and second portion 210, as illustrated in FIG. 4B. The commissure tab assembly 228 can be constructed similar to commissure tab assembly 150 discussed above and illustrated in FIGS. 3A-3B. For example, commissure tab assembly 228 can include a pair of adjacent leaflets 222a, 222b coupled together at tab portions 224a, 224b. Similar to commissure tab assembly 150, a covering member may be coupled to the ends of the tab portions 224a, 224b, and optional reinforcing members may be coupled to the tab portions 224a, 224b at a location adjacent to the inner side 206 of commissure support post 200. However, for simplicity in illustration, only the leaflet components of the commissure tab assembly 228 are shown in FIG. 4B.

In the illustrated embodiment of FIG. 4B, the commissure tab assembly 228 can be coupled to the commissure support post in a manner similar to that discussed above and illustrated in FIGS. 3B-C. For example, the free ends of tab portions 224a, 224b of the commissure tab assembly 228 can be flared in the circumferential direction 218 and then wrapped around at least the inner side 206 and circumferential sides 204a, 204b of the commissure support post 200. The free ends of the tab portions 224a, 224b can connected to each other with sutures as shown in FIGS. 3B-C. Edges of the leaflets 222a, 222b can be disposed adjacent to, and optionally in contact with, a bottom surface of the protrusion 230. The protrusion 230 thus restricts the commissure tab assembly 228 (and thereby the leaflets 222a, 222b) from moving along the longitudinal direction 220 toward the first end portion 208.

In some embodiments, such as illustrated in FIG. 4B, one or more sutures 226a, 226b can extend through aperture 214 of protrusion 230 and a respective portion of the commissure tab assembly 228, so as to secure the commissure tab assembly 228 to the protrusion 230. For example, sutures 226a, 226b can pass through the commissure tab assembly 228 at a portion where the optional reinforcing members are disposed, at a part of the leaflet tab portions 224a, 224b adjacent to and spaced outward along the circumferential direction 218 from a location where the reinforcing members are disposed, or at any other location along the commissure tab assembly 228. Sutures 226a, 226b can thus prevent, or at least restrict, rotation of the commissure tab assembly 228 around the commissure support post 200, thereby maintaining a central alignment of the leaflet assembly within the annular frame 12.

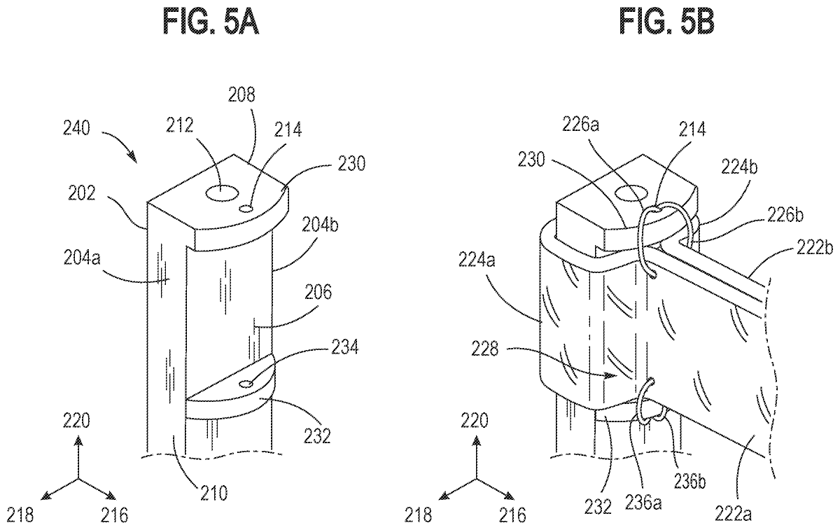

In some embodiments, the commissure support post can be provided with one or more additional protrusions at an opposite axial end of the commissure tab assembly so as to further restrict motion of the commissure tab assembly along the longitudinal direction. Referring to FIG. 5A, a second example of a commissure support post 240 is illustrated. Commissure support post 240 has a similar structure to commissure support post 200 of FIG. 4A but with at least one additional protrusion 232 axially spaced from the protrusion 230 at a second portion 210 of the post 240. Depending on the overall length of the post, the second portion 210 can be an end portion of the post 240 or an intermediate portion of the post (in which case the protrusion 232 is located along an intermediate portion of the post). In the following description, second portion 210 is referred to as an end portion of the post; however, it should be understood that the protrusion 232 can be located at any location along the post axially spaced from the protrusion 230. In the illustrated embodiment of FIG. 5A, both protrusion 230 and 232 project inward along radial direction 216 from the inner side 206 of the commissure support post 240, such that at least a portion of each protrusion 230, 232 is closer to a center of the frame 12 than the commissure support post 240.

In the illustrated embodiment of FIGS. 5A-5B, protrusion 230 includes at least one aperture or through-hole 214, through which a suture or other attachment means can be passed. Similarly, protrusion 232 includes at least one aperture or through-hole 234, through which a suture or other attachment means can be passed. In some embodiments, each protrusion 230, 232 is provided with multiple respective apertures 214, 234, each of which is constructed to receive passage of a respective suture or other attachment means therethrough. Although apertures 214, 234 are shown in FIG. 5A as extending along longitudinal direction 220 with openings at top and bottom surfaces of the protrusions 230, 232, in some embodiments either or both of apertures 214, 234 can instead extend along the circumferential direction 218 with openings at circumferential surfaces of the respective protrusion 230, 232.

As illustrated in FIG. 5B, the commissure tab assembly 228 can be coupled to the commissure support post 240 between first end portion 208 and second portion 210, in particular, between protrusion 230 and protrusion 232. Construction of the commissure tab assembly 228 and assembly thereof to the commissure support post 240 may be similar to that described above with respect to FIG. 4B. For example, the free ends of tab portions 224a, 224b of the commissure tab assembly 228 can be flared in circumferential direction 218 and then wrapped around at least the inner side 206 and circumferential sides 204a, 204b of the commissure support post 240. The free ends of the tab portions 224a, 224b can connected to each other with sutures as shown in FIGS. 3B-C.

Edges of the leaflets 222a, 222b can be disposed adjacent to, and optionally in contact with, a bottom surface of the protrusion 230. Similarly, opposite edges of the leaflets 222a, 222b can be disposed adjacent to, and optionally in contact with, a top surface of the protrusion 232. Thus, the protrusion 230 thus restricts the commissure tab assembly 228 (and thereby leaflets 222a, 222b) from moving along the longitudinal direction 220 toward the first end portion 208, while protrusion 232 restricts the commissure tab assembly 228 (and thereby leaflets 222a, 222b) from moving along the longitudinal direction 220 toward the second portion 210.

In some embodiments, such as illustrated in FIG. 5B, one or more sutures 226a, 226b can extend through aperture 214 of protrusion 230 and a respective portion of the commissure tab assembly 228, so as to secure the commissure tab assembly 228 to the protrusion 230. Alternatively or additionally, one or more sutures 236a, 236b can extend through aperture 234 of protrusion 232 and respective portion of the commissure tab assembly 228, so as to secure the commissure tab assembly 228 to the protrusion 232. For example, sutures 226a, 226b, 236a, 236b can pass through the commissure tab assembly 228 at a portion where the reinforcing members are disposed, at a part of the leaflet tab portions 224a, 224b adjacent to and spaced along the circumferential direction 218 from a location where the reinforcing members are disposed, or at any other location along the commissure tab assembly 228. Sutures 226a, 226b, 236a, and/or 236b can thus prevent, or at least restrict, rotation of the commissure tab assembly 228 around the commissure support post 240, thereby maintaining a central alignment of the leaflet assembly within the annular frame 12.

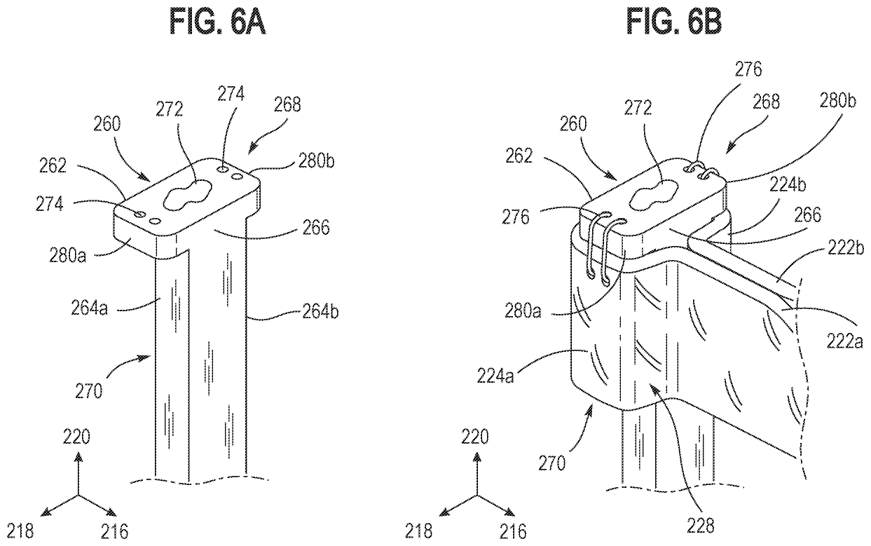

In some embodiments, the commissure support post can be provided with one or more protrusions that project circumferentially rather than radially. Referring to FIG. 6A, a third example of a commissure support post 260 is illustrated. Commissure support post 260 has a similar structure to commissure support post 200 of FIG. 4A. For example, commissure support post 260 has a radially inner side 266, which faces inward toward a centerline of the annular frame, and a radially outer side 262, which faces outward toward an exterior of the annular frame and toward the patient's anatomy when the prosthetic heart valve is implanted. A pair of opposing sides 264a, 264b connects the outer side 262 to the inner side 266 at respective edges thereof.

However, instead of protrusion 230 that projects inward along radial direction 216, first end portion 268 of commissure support post 260 includes side protrusions 280a, 280b that project along circumferential direction 218 away from respective sides 264a, 264b of the commissure support post 260. At a second portion 270 remote from the first end portion 268, the commissure support post 260 does not include any protrusions. In some embodiments, the commissure support post 260 can have one or more longitudinally-extending internal lumens 272 disposed at a location spaced circumferentially between protrusions 280a, 280b. For example, when the commissure support post 260 is a part of an actuator 80 of the annular frame 12, internal lumen 272 may be an actuation lumen or locking lumen.

Although FIG. 6A illustrates a particular location of side protrusions 280a, 280b coincident with an end of the commissure support post 260, in some embodiments the side protrusions 280a, 280b can be located at other positions along the length of the commissure support post 260, such that the commissure support post 260 extends both above and below the side protrusions 280a, 280b along longitudinal direction 220. In such embodiments, upper surfaces of side protrusions 280a, 280b would not be coincident or coplanar with an upper surface of the commissure support post 260; however, each side protrusion 280a, 280b would be disposed closer to first end portion 268 than the second portion 270.

In some embodiments, each side protrusion 280a, 280b includes at least one aperture or through-hole 274, through which a suture or other attachment means can be passed. In the illustrated embodiment of FIGS. 6A-6B, each side protrusion 280a, 280b is provided with multiple apertures 274, each of which is constructed to receive passage of a respective suture or other attachment means therethrough. Although each aperture 274 is shown in FIG. 6A as extending along longitudinal direction 220 with openings at top and bottom surfaces of the respective side protrusion 280a, 280b, in some embodiments each aperture 274 can instead extend along the radial direction 216 with openings at radially-inner and radially-outer side surfaces of the respective side protrusion 280a, 280b.

As illustrated in FIG. 6B, the commissure tab assembly 228 can be coupled to the commissure support post 260 between first end portion 268 and second portion 270. Construction of the commissure tab assembly 228 and assembly thereof to the commissure support post 260 may be similar to that described above with respect to FIG. 4B. For example, the free ends of tab portions 224a, 224b of the commissure tab assembly 228 can be flared in circumferential direction 218 and then wrapped around at least the inner side 266 and circumferential sides 264a, 264b of the commissure support post 260. The free ends of the tab portions 224a, 224b can connected to each other with sutures as shown in FIGS. 3B-C. An edge of tab portion 224a can be disposed adjacent to, and optionally in contact with, a bottom surface of side protrusion 280a, while an edge of tab portion 224b can be disposed adjacent to, and optionally in contact with, a bottom surface of side protrusion 280b. The side protrusions 280a, 280b thus restrict the commissure tab assembly 228 (and thereby the leaflets 222a, 222b) from moving along the longitudinal direction 220 toward the first end portion 268.

In some embodiments, such as illustrated in FIG. 6B, one or more sutures 276 can extend through apertures 274 of each side protrusion 280a, 280b and a respective portion of the commissure tab assembly 228, so as to secure the commissure tab assembly 228 to protrusions 280a, 280b. For example, sutures 276 can pass through the commissure tab assembly 228 at a portion contacting the respective side 264a, 264b of commissure support post 260. Sutures 276 can thus prevent, or at least restrict, rotation of the commissure tab assembly 228 around the commissure support post 260, thereby maintaining a central alignment of the leaflet assembly within the annular frame 12.

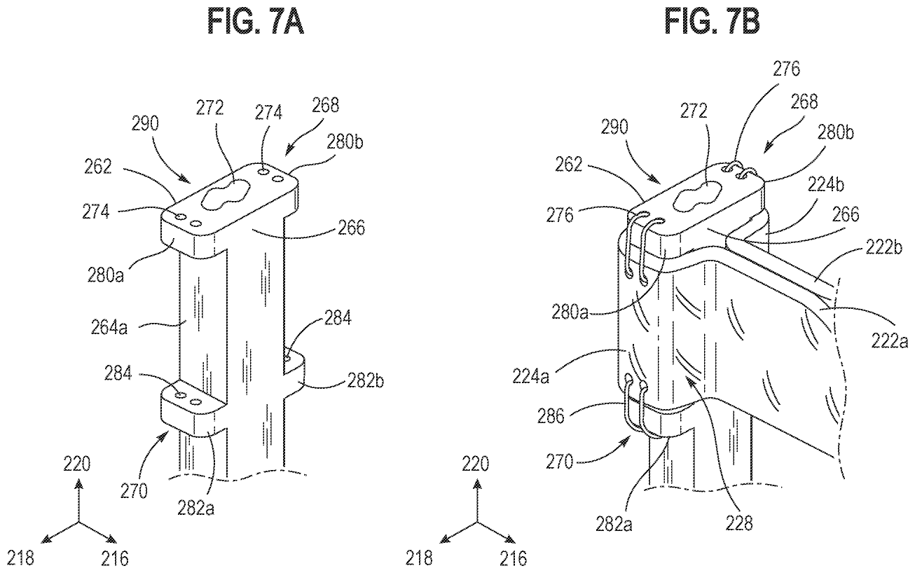

In some embodiments, the commissure support post can be provided with one or more additional side protrusions at an opposite axial end of the commissure tab assembly so as to further restrict motion of the commissure tab assembly along the longitudinal direction. Referring to FIG. 7A, a fourth example of a commissure support post 290 is illustrated. Commissure support post 290 has a similar structure to commissure support post 260 of FIG. 6A but with at least one additional pair of side protrusions 282a, 282b axially spaced from the side protrusions 280a, 280b, such as at a second portion 270 of the post. In the illustrated embodiment of FIG. 7A, each of the side protrusions 280a, 280b, 282a, 282b project along circumferential direction 218 away from respective sides 264a, 264b of the commissure support post 290.

In some embodiments, each side protrusion 280a, 280b includes at least one aperture or through-hole 274 through which a suture or other attachment means can be passed, and each side protrusion 282a, 282b includes at least one aperture or through-hole 284 through which a suture or other attachment means can be passed. In the illustrated embodiment of FIGS. 7A-7B, each side protrusion 280a, 280b is provided with multiple apertures 274, each of which is constructed to receive passage of a respective suture or other attachment means therethrough, and each side protrusion 282a, 282b is provided with multiple apertures 284, each of which is constructed to receive passage of a respective suture or other attachment means therethrough. Although each aperture 274, 284 is shown in FIG. 7A as extending along longitudinal direction 220 with openings at top and bottom surfaces of the respective side protrusion 280a, 280b, 282a, 282b, in some embodiments each aperture 274, 284 can instead extend along the radial direction 216 with openings at radially-inner and radially-outer side surfaces of the respective side protrusion 280a, 280b, 282a, 282b.

As illustrated in FIG. 7B, the commissure tab assembly 228 can be coupled to the commissure support post 290 between first end portion 268 and second portion 270, in particular, between side protrusions 280a, 280b at one end and side protrusions 282a, 282b at another end. Construction of the commissure tab assembly 228 and assembly thereof to the commissure support post 290 may be similar to that described above with respect to FIG. 4B. For example, the free ends of tab portions 224a, 224b of the commissure tab assembly 228 can be flared in circumferential direction 218 and then wrapped around at least the inner side 266 and circumferential sides 264a, 264b of the commissure support post 290. The free ends of the tab portions 224a, 224b can connected to each other with sutures as shown in FIGS. 3B-C.

An edge of tab portion 224a can be disposed adjacent to, and optionally in contact with, a bottom surface of side protrusion 280a, while an edge of tab portion 224b can be disposed adjacent to, and optionally in contact with, a bottom surface of side protrusion 280b. Similarly, an opposite edge of tab portion 224a can be disposed adjacent to, and optionally in contact with, a top surface of side protrusion 282a, while an opposite edge of tab portion 224b can be disposed adjacent to, and optionally in contact with, a top surface of side protrusion 282b. Thus, the side protrusions 280a, 280b restrict the commissure tab assembly 228 (and thereby the leaflets 222a, 222b) from moving along the longitudinal direction 220 toward the first end portion 268, while side protrusion 282a, 282b restrict the commissure tab assembly 228 (and thereby leaflets 222a, 22b) from moving along the longitudinal direction 220 toward the second portion 270.

In some embodiments, such as illustrated in FIG. 7B, one or more sutures 276 can extend through apertures 274 of each side protrusion 280a, 280b and a respective portion of the commissure tab assembly 228, so as to secure the commissure tab assembly 228 to protrusions 280a, 280b. Alternatively or additionally, one or more sutures 286 can extend through aperture 284 of side protrusion 282a, 282b and a respective portion of the commissure tab assembly 228, so as to secure the commissure tab assembly 228 to the protrusions 282a, 282b. For example, sutures 276, 286 can pass through the commissure tab assembly 228 at portions contacting the respective sides 264a, 264b of commissure support post 290. Sutures 276, 286 can thus prevent, or at least restrict, rotation of the commissure tab assembly 228 around the commissure support post 290, thereby maintaining a central alignment of the leaflet assembly within the annular frame 12.

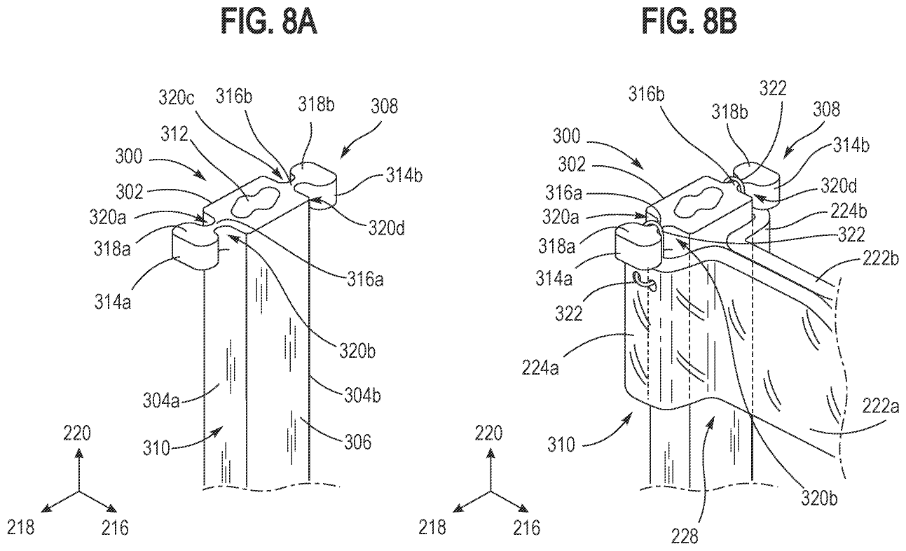

In some embodiments, the one or more protrusions of the commissure support post can form slots or recesses through which sutures can pass instead of through-holes or apertures. Referring to FIG. 8A, a fifth example of such a commissure support post 300 is illustrated. Commissure support post 300 has a similar structure to commissure support post 260 of FIG. 6A. For example, commissure support post 300 has a radially inner side 306, which faces inward toward a centerline of the annular frame, and a radially outer side 302, which faces outward toward an exterior of the annular frame and toward the patient's anatomy when the prosthetic heart valve is implanted. A pair of opposing sides 304a, 304b connects the outer side 302 to the inner side 306 at respective edges thereof.

However, instead of side protrusions 280a, 280b with respective apertures 274, first end portion 308 of commissure support post 300 includes side protrusions 314a, 314b without any apertures. Rather, each side protrusion 314a, 314b includes a respective narrower width neck portion 316a, 316b and a larger width end portion 318a, 318b. Each side protrusion 314a, 314b projects along circumferential direction 218 away from respective sides 304a, 304b of the commissure support post 300. Thus, a pair of radially-extending slots 320a, 320b, which are separated from each other by neck portion 316a, are formed between protrusion 314a and side 304a of the commissure support 300 (e.g., a central portion of commissure support 300). Similarly, a pair of radially-extending slots 320c, 320d, which are separated from each other by neck portion 316b, are formed between protrusion 314b and side 304b of the commissure support 300 (e.g., a central portion of commissure support 300). Alternatively, each end portion 318a, 318b can be arranged with respect to the neck portion 316a, 316b such that only a single slot is formed between the protrusion 314a, 314b and the respective side 304a, 304b of the commissure support post 300.

At a second end portion 310 remote from the first end portion 308, the commissure support post 300 does not include any protrusions. In some embodiments, the commissure support post 300 can have one or more longitudinally-extending internal lumens 312 disposed at a location spaced circumferentially between protrusions 314a, 314b. For example, when the commissure support post 300 is a part of an actuator 80 of the annular frame 12, internal lumen 312 may be an actuation lumen or locking lumen.

Although FIG. 8A illustrates a particular location of side protrusions 314a, 314b coincident with an end of the commissure support post 300, in some embodiments the side protrusions 314a, 314b can be located at other positions along the length of the commissure support post 300, such that the commissure support post 300 extends both above and below the side protrusions 314a, 314b along longitudinal direction 220. In such embodiments, upper surfaces of side protrusions 314a, 314b would not be coincident or coplanar with an upper surface of the commissure support post 300; however, each side protrusion 314a, 314b would be disposed closer to first end portion 308 than the second end portion 310.

As illustrated in FIG. 8B, the commissure tab assembly 228 can be coupled to the commissure support post 300 between first end portion 308 and second end portion 310. Construction of the commissure tab assembly 228 and assembly thereof to the commissure support post 300 may be similar to that described above with respect to FIG. 4B. For example, the free ends of tab portions 224a, 224b of the commissure tab assembly 228 can be flared in circumferential direction 218 and then wrapped around at least the inner side 306 and circumferential sides 304a, 304b of the commissure support post 300. The free ends of the tab portions 224a, 224b can connected to each other with sutures as shown in FIGS. 3B-C. An edge of tab portion 224a can be disposed adjacent to, and optionally in contact with, a bottom surface of side protrusion 314a (e.g., neck portion 316a), while an edge of tab portion 224b can be disposed adjacent to, and optionally in contact with, a bottom surface of side protrusion 314b (e.g., neck portion 316b). The side protrusions 314a, 314b thus restrict the commissure tab assembly 228 (and thereby the leaflets 222a, 222b) from moving along the longitudinal direction 220 toward the first end portion 308.

In some embodiments, such as illustrated in FIG. 8B, one or more sutures 322 can extend through slots formed by each side protrusion 314a, 314b and can wrap at least partially around respective neck portions 316a, 316b to secure the commissure tab assembly 228 to the protrusions 314a, 314b. For example, suture 322 can extend through slot 320a, wrap over neck portion 316a, extend through slot 320b, and pass through leaflet tab 224a, which contacts side 304a of commissure support post 300. Similarly, another suture 322 (or the same suture, not shown) can extend through slot 320c, wrap over neck portion 316b, extend through slot 320d, and pass through leaflet tab 224b, which contacts side 304b of commissure support post 300. Sutures 322 can thus prevent, or at least restrict, rotation of the commissure tab assembly 228 around the commissure support post 300, thereby maintaining a central alignment of the leaflet assembly within the annular frame 12.

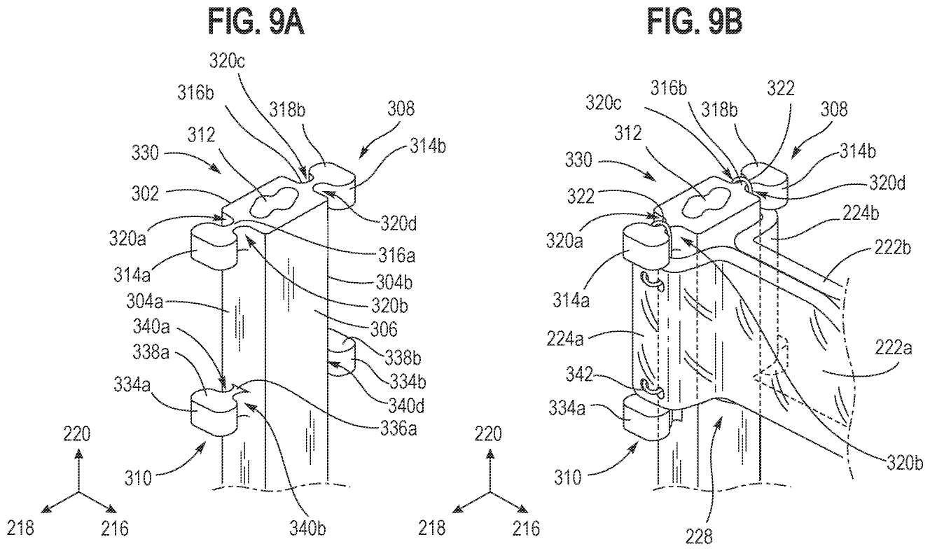

In some embodiments, the commissure support post can be provided with one or more additional side protrusions at an opposite axial end of the commissure tab assembly so as to further restrict motion of the commissure tab assembly along the longitudinal direction. Referring to FIG. 9A, a sixth example of a commissure support post 330 is illustrated. Commissure support post 330 has a similar structure to commissure support post 300 of FIG. 8A but with at least one additional pair of side protrusions 334a, 334b at a second end portion 310 thereof.

Each side protrusion 334a, 334b includes a respective narrower width neck portion and a larger width end portion 338a, 338b, and projects along circumferential direction 218 away from respective sides 304a, 304b of the commissure support post 330 (e.g., a central portion of commissure support post 330). Thus, another pair of radially-extending slots 340a, 340b, which are separated from each other by neck portion 336a, are formed between protrusion 334a and side 304a of the commissure support post 330 (e.g., a central portion of commissure support post 330). Similarly, a pair of radially-extending slots (only 340d being visible in FIG. 9A), which are separated from each other by a neck portion (not visible in FIGS. 9A-9B), are formed between protrusion 334b and side 304b of the commissure support post 330 (e.g., a central portion of commissure support post 330). Alternatively, each end portion 338a, 338b can be arranged with respect to the corresponding neck portion such that only a single slot is formed between the protrusion 334a, 334b and the respective side 304a, 304b of the commissure support post 330.

As illustrated in FIG. 9B, the commissure tab assembly 228 can be coupled to the commissure support post 330 between first end portion 308 and second end portion 310, in particular, between side protrusions 314a, 314b at one end and side protrusions 334a, 334b at another end. Construction of the commissure tab assembly 228 and assembly thereof to the commissure support post 330 may be similar to that described above with respect to FIG. 4B. For example, the free ends of tab portions 224a, 224b of the commissure tab assembly 228 can be flared in circumferential direction 218 and then wrapped around at least the inner side 306 and circumferential sides 304a, 304b of the commissure support post 330. The free ends of the tab portions 224a, 224b can connected to each other with sutures as shown in FIGS. 3B-C.

An edge of tab portion 224a can be disposed adjacent to, and optionally in contact with, a bottom surface of side protrusion 314a (e.g., neck portion 316a), while an edge of tab portion 224b can be disposed adjacent to, and optionally in contact with, a bottom surface of side protrusion 314b (e.g., neck portion 316b). Similarly, an opposite edge of tab portion 224a can be disposed adjacent to, and optionally in contact with, a top surface of side protrusion 334a (e.g., neck portion 336a), while an opposite edge of tab portion 224b can be disposed adjacent to, and optionally in contact with, a top surface of side protrusion 334b (e.g., neck portion between end portion 338b and side 304b). Thus, the side protrusions 314a, 314b restrict the commissure tab assembly 228 (and thereby the leaflets 222a, 222b) from moving along the longitudinal direction 220 toward the first end portion 308, while side protrusion 334a, 334b restrict the commissure tab assembly 228 (and thereby leaflets 222a, 222b) from moving along the longitudinal direction 220 toward the second end portion 310.

As discussed above with respect to FIG. 8B, one or more sutures 322 can extend through slots 320a-320d formed by side protrusions 314a, 314b to secure the commissure tab assembly thereto. In some embodiments, such as illustrated in FIG. 9B, one or more sutures 342 can extend through slots formed by each side protrusion 334a, 334b and can wrap at least partially around respective neck portions to secure the commissure tab assembly 228 to the protrusions 334a, 334b. For example, suture 342 can extend through radially-outer slot 340a, wrap under neck portion 336a, extend through radially-inner slot 340b, and pass through leaflet tab 224a, which contacts side 304a of commissure support post 330. Similarly, another suture (or the same suture 342) can extend through a radially-outer slot, wrap under neck portion (between end portion 338b and side 304b), extend through radially-inner slot 320d, and pass through leaflet tab 224b, which contacts side 304b of commissure support post 330. Sutures 342 can thus further prevent, or at least further restrict, rotation of the commissure tab assembly 228 around the commissure support post 330, thereby maintaining a central alignment of the leaflet assembly within the annular frame 12.

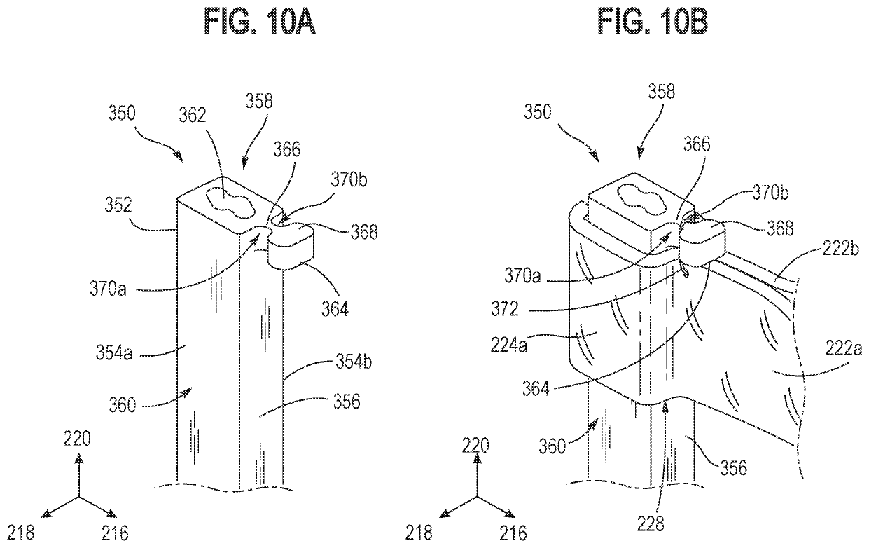

In some embodiments, the commissure support can be provided with one or more protrusions that form circumferentially-extending slots instead of radially-extending slots and that project radially rather than circumferentially. Referring to FIG. 10A, a seventh example of a commissure support post 350 is illustrated. Commissure support post 350 has a similar structure to commissure support post 300 of FIG. 8A. For example, commissure support post 350 has a radially inner side 356, which faces inward toward a centerline of the annular frame, and a radially outer side 352, which faces outward toward an exterior of the annular frame and toward the patient's anatomy when the prosthetic heart valve is implanted. A pair of opposing sides 354a, 354b connects the outer side 352 to the inner side 356 at respective edges thereof.

However, instead of protrusions 314a, 314b that project circumferentially, first end portion 358 of commissure support post 350 includes protrusion 364 that projects inward along radial direction 216 and away from inner side 356 of the commissure support post 350. At a second portion 360 remote from the first end portion 358, the commissure support post 350 does not include any protrusions. In some embodiments, the commissure support post 350 can have one or more longitudinally-extending internal lumens 362 disposed at a location spaced radially outward from protrusion 364. For example, when the commissure support post 350 is a part of an actuator 80 of the annular frame 12, internal lumen 362 may be an actuation lumen or locking lumen.

Protrusion 364 includes a narrower-width neck portion 366 and a larger-width end portion 368 and projects along radial direction 216 away from inner side 356 of the commissure support post 350. Thus, a pair of circumferentially-extending slots 370a, 370b, which are separated from each other by neck portion 366, are formed between protrusion 364 and side 356 of the commissure support post 350 (e.g., a central portion of commissure support post 350). Alternatively, end portion 368 can be arranged with respect to the neck portion 366 such that only a single slot is formed between the protrusion 364 and side 356 of the commissure support post 350.

Although FIG. 10A illustrates a particular location of protrusion 364 coincident with an end of the commissure support post 350, in some embodiments, protrusion 364 can be located at other positions along the length of the commissure support post 350, such that the commissure support post 350 extends both above and below the protrusion 364 along longitudinal direction 220. In such embodiments, upper surface of protrusions 364 would not be coincident or coplanar with an upper surface of the commissure support post 350; however, protrusion 364 would be disposed closer to first end portion 358 than the second portion 360.

As illustrated in FIG. 10B, the commissure tab assembly 228 can be coupled to the commissure support post 350 between first end portion 358 and second portion 360. Construction of the commissure tab assembly 228 and assembly thereof to the commissure support post 350 may be similar to that described above with respect to FIG. 4A. For example, the free ends of tab portions 224a, 224b of the commissure tab assembly 228 can be flared in the circumferential direction 218 and then wrapped around at least the inner side 356 and circumferential sides 354a, 354b of the commissure support post 350. The free ends of the tab portions 224a, 224b can connected to each other with sutures as shown in FIGS. 3B-C. Edges of the leaflets 222a, 222b can be disposed adjacent to, and optionally in contact with, a bottom surface of the protrusion 364 (e.g., neck portion 366 and/or end portion 368). The protrusion 364 thus restricts the commissure tab assembly 228 (and thereby the leaflets 222a, 222b) from moving along the longitudinal direction 220 toward the first end portion 358.

In some embodiments, such as illustrated in FIG. 10B, one or more sutures 372 can extend through slots formed by protrusion 364 and can wrap at least partially around neck portion 366 to secure the commissure tab assembly 228 to the protrusion 364. For example, suture 372 can extend through left slot 370a, wrap over neck portion 366, extend through right slot 370b, and pass through a portion of commissure tab assembly 228. For example, sutures 372 can pass through the commissure tab assembly 228 at a portion where the optional reinforcing members are disposed, at a part of the leaflet tab portions 224a, 224b facing or contacting inner side 356, or at any other location along the commissure tab assembly 228. Sutures 372 can thus prevent, or at least restrict, rotation of the commissure tab assembly 228 around the commissure support post 350, thereby maintaining a central alignment of the leaflet assembly within the annular frame 12.

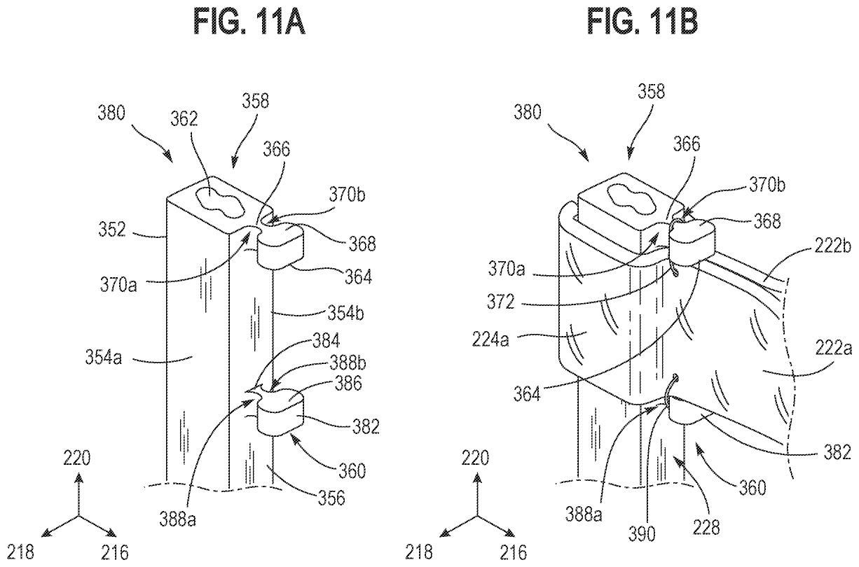

In some embodiments, the commissure support post can be provided with one or more additional protrusions at an opposite axial end of the commissure tab assembly so as to further restrict motion of the commissure tab assembly along the longitudinal direction. Referring to FIG. 11A, an eighth example of a commissure support post 380 is illustrated. Commissure support post 380 has a similar structure to commissure support post 350 of FIG. 10A but with at least one additional protrusion 382 axially spaced from the protrusion 364, such as at a second portion 360 of the post. Additional protrusion 382 includes a narrower-width neck portion 384 and a larger-width end portion 386, and projects inward along radial direction 216 and away from inner side 356 of the commissure support post 380. Thus, another pair of circumferentially-extending slots 388a, 388b, which are separated from each other by neck portion 384, are formed between protrusion 382 and side 356 of the commissure support post 380 (e.g., a central portion of commissure support post 380). Alternatively, the end portion 386 can be arranged with respect to neck portion 384 such that only a single slot is formed between the protrusion 382 and inner side 356 of the commissure support post 380.

As illustrated in FIG. 11B, the commissure tab assembly 228 can be coupled to the commissure support post 380 between first end portion 358 and second portion 360, in particular, between protrusion 364 at one end and protrusion 382 at another end. Construction of the commissure tab assembly 228 and assembly thereof to the commissure support post 330 may be similar to that described above with respect to FIG. 4B. For example, the free ends of tab portions 224a, 224b of the commissure tab assembly 228 can be flared in circumferential direction 218 and then wrapped around at least the inner side 356 and circumferential sides 354a, 354b of the commissure support post 380. The free ends of the tab portions 224a, 224b can connected to each other with sutures as shown in FIGS. 3B-C. Edges of the leaflets 222a, 222b can be disposed adjacent to, and optionally in contact with, a bottom surface of protrusion 364 (e.g., neck portion 366 and/or end portion 368). Similarly, opposite edges of leaflets 222a, 222b can be disposed adjacent to, and optionally in contact with, a top surface of protrusion 382 (e.g., neck portion 384 and/or end portion 386). Thus, protrusion 364 restricts the commissure tab assembly 228 (and thereby the leaflets 222a, 222b) from moving along the longitudinal direction 220 toward the first end portion 358, while protrusion 382 restricts the commissure tab assembly 228 (and thereby leaflets 222a, 222b) from moving along the longitudinal direction 220 toward the second portion 360.

One or more sutures 372 can extend through slots 370a, 370b formed by protrusion 364 and can wrap at least partially around neck portion 366 to secure the commissure tab assembly 228 to the protrusion 364. Alternatively or additionally, one or more sutures 390 can extend through slots 388a, 388b formed by protrusion 382 and can wrap at least partially around neck portion 384 to secure the commissure tab assembly 228 to protrusions 382. For example, suture 390 can extend through left slot 388a, wrap under neck portion 384, extend through right slot 388b, and pass through a portion of commissure tab assembly 228. For example, sutures 390 can past through the commissure tab assembly 228 at a portion where the optional reinforcing members are disposed, at a part of the leaflet tab portions 224a, 224b facing or contacting inner side 356, or at any other location along the commissure tab assembly 228. Sutures 372, 390 can thus prevent, or at least restrict, rotation of the commissure tab assembly 228 around the commissure support post 380, thereby maintaining a central alignment of the leaflet assembly within the annular frame 12.

Although particular shapes and configurations for the commissure support post have been illustrated in the figures and described in the examples above, embodiments of the disclosed subject matter are not limited thereto. Indeed, in some embodiments, the commissure support post may have a cross-sectional geometry (e.g., in a plane perpendicular to the longitudinal axis of the annular frame) that is circular, oval, hexagonal, octagonal, or any other shape. However, in some embodiments, it may be preferable that the corners and/or edges of the commissure support post be rounded (e.g., have a radius) or chamfered, for example, to avoid catching or tearing of sutures or leaflets. Moreover, although particular shapes and configurations for the protrusions of the commissure support post have been illustrated in the figures and described in the examples above, different shapes and/or configurations are also possible according to one or more contemplated embodiments. For example, the radially-extending protrusion can have a width along the circumferential direction that is less than that of the commissure support post, and/or the circumferentially-extending protrusion can have a width along the radial direction that is less than that of the commissure support post. The sizes and shapes of each commissure support post and protrusions thereon may take into account any required clearance, in particular, when the heart valve is in a fully collapsed or crimped state, for example, to allow for a minimized cross-sectional diameter of the heart valve for implantation into a patient and/or to avoid interfering with operation of various components of the valve, e.g., operation of the leaflets once implanted.

Although a particular number of protrusions for the commissure support post has been illustrated in the figures and described in the examples above, the commissure support post in embodiments of the disclosed subject matter can include fewer or additional protrusions. For example, a commissure support post with multiple circumferentially-extending protrusions may instead have a single circumferentially-extending protrusion. In another example, the single radially-extending protrusion of a commissure support post may be replaced by multiple radially-extending protrusions, with a radially-extending gap therebetween. In yet another example, the single circumferentially-extending protrusion at one or both sides of a commissure support post may be replaced by multiple circumferentially-extending protrusions, with a circumferentially-extending gap therebetween.

Although a particular number, size, and shape of apertures and slots has been illustrated in the figures and described in the example above, other numbers, sizes, and/or shapes are also possible according to one or more contemplated embodiments. For example, multiple apertures in a projection may be combined together into a single larger aperture or slot. In general, the sizing of apertures may be such that a needle carrying the suture is capable of passing therethrough. In contrast, since slots remain open at one end, the width of the slot may be based on the cross-sectional dimension of the suture passing therethrough rather than the needle, which has a diameter larger than the cross-sectional dimension of the suture.

In some embodiments, features separately illustrated the different figures or described above as separate examples may nevertheless be combined together. For example, in some embodiments, a commissure support post can have both radially-extending protrusions and circumferentially-extending protrusions at the same end. Alternatively or additionally, the commissure support post can have one type of protrusion at the first end (e.g., a radially-extending protrusion) and a different type of protrusion at the second end (e.g., a pair of circumferentially-extending protrusions). In some embodiments, a protrusion of the commissure support post may have an aperture for passing a suture therethrough and may also have a narrow neck portion for forming a slot for passing the same or a different suture therethrough. In some embodiments, radially-extending and circumferentially-extending protrusions at a same end of the commissure support post can be combined together in single continuous geometry (e.g., with a cross-sectional shape of the commissure support post being completely contained within a cross-sectional shape of the single protrusion in a plan view).

In some embodiments, protrusions may be reduced in size or eliminated altogether in favor of apertures directly formed in a body of the commissure support post. For example, a commissure support post can include a hole or aperture that extends through an entire radial or circumferential width of the post. One or more sutures can then be passed through the aperture to secure the commissure tab assembly in place on the commissure support post. However, in embodiments where the commissure support post may be part of the locking or actuator assembly, such through-holes should be positioned to avoid interfering with the locking or actuation mechanisms (e.g., by avoiding internal lumen 212 in FIG. 4A). Alternatively or additionally, the commissure support post can include a curving internal pathway with a first opening on an exposed upper surface of the post and a second opening on a radially-inner or circumferential-side wall. A suture can extend through the internal pathway and through the commissure tab assembly to secure the assembly in position on the post.

### Additional Description of Embodiments of Interest

Clause 1. A prosthetic heart valve, comprising:

an annular frame that is radially collapsible and expandable between a radially-compressed configuration and a radially-expanded configuration, the annular frame having an inflow end and an outflow end separated from the inflow end along an axial direction of the frame, the annular frame comprising a plurality of commissure support posts; and

a valvular structure supported within the annular frame and comprising a plurality of leaflets, each leaflet having a pair of tabs, the tabs of adjacent leaflets being paired together to form a commissure tab assembly,

wherein there is one commissure support post for each commissure tab assembly,

each commissure tab assembly is coupled to the corresponding commissure support post with one tab wrapped at least partially around the commissure support post in a first direction and the other tab wrapped at least partially around the commissure support post in a second direction,

each commissure support post has a first portion with a pair of first protrusions that project in opposite directions along a circumferential direction of the annular frame,

for each commissure support post, the corresponding commissure tab assembly is disposed along the axial direction between the first portion and the inflow end of the annular frame, and

for each commissure support post, the pair of first protrusions is arranged along the axial direction so as to restrain axial motion of the respective commissure tab assembly in a downstream direction.

Clause 2. The prosthetic heart valve of clause 1, wherein, for each commissure support post, the first portion is an end portion thereof closest to the outflow end of the annular frame.

Clause 3. The prosthetic heart valve of any of clauses 1-2, wherein each commissure post has a second portion with a pair of second protrusions that project in opposite directions along the circumferential direction of the annular frame.

Clause 4. The prosthetic heart valve of clause 3, wherein, for each commissure support post: the corresponding commissure tab assembly is disposed along the axial direction between the first and second portions, and the pair of second protrusions is arranged along the axial direction so as to restrain axial motion of the respective commissure tab assembly in an upstream direction.

Clause 5. The prosthetic heart valve of any of clauses 3-4, wherein, for each commissure support post, the second portion is between the first portion and an end portion of the commissure support post closest to the inflow end of the annular frame.

Clause 6. The prosthetic heart valve of any of clauses 3-5, wherein, for each commissure support post: each second protrusion includes at least one aperture, and the tabs of the corresponding commissure tab assembly are coupled to the commissure support post via one or more sutures that pass through the apertures of the pair of second protrusions.

Clause 7. The prosthetic heart valve of clause 6, wherein, for each second protrusion, each aperture is a hole or opening that extends along the axial direction through a thickness of the respective second protrusion.

Clause 8. The prosthetic heart valve of any of clauses 1-7, wherein, for each commissure support post: each first protrusion includes at least one aperture, and the tabs of the corresponding commissure tab assembly are coupled to the commissure support post via one or more sutures that pass through the apertures of the pair of first protrusions.

Clause 9. The prosthetic heart valve of clause 8, wherein, for each first protrusion, each aperture is a hole or opening that extends along the axial direction through a thickness of the respective first protrusion.

Clause 10. A prosthetic heart valve, comprising:

an annular frame that is radially collapsible and expandable between a radially-compressed configuration and a radially-expanded configuration, the annular frame having an inflow end and an outflow end separated from the inflow end along an axial direction of the frame, the annular frame comprising a plurality of commissure support posts; and

a valvular structure supported within the annular frame and comprising a plurality of leaflets, each leaflet having a pair of tabs, the tabs of adjacent leaflets being paired together to form a commissure tab assembly,

wherein there is one commissure support post for each commissure tab assembly,

each commissure tab assembly is coupled to the corresponding commissure support post with one tab wrapped at least partially around the commissure support post in a first direction and the other tab wrapped at least partially around the commissure support post in a second direction,

each commissure support post has a first portion with a first protrusion that projects along a radial direction of the annular frame toward an interior of the annular frame,

for each commissure support post, the corresponding commissure tab assembly is disposed along the axial direction between the first portion and the inflow end of the annular frame, and

for each commissure support post, the first protrusion is arranged along the axial direction so as to restrain axial motion of the respective commissure tab assembly in a downstream direction.

Clause 11. The prosthetic heart valve of clause 10, wherein, for each commissure support post, the first portion is an end portion thereof closest to the outflow end of the annular frame.

Clause 12. The prosthetic heart valve of any of clauses 10-11, wherein each commissure post has a second portion with a second protrusion that projects along the radial direction toward the interior of the annular frame.

Clause 13. The prosthetic heart valve of clause 12, wherein, for each commissure support post: the corresponding commissure tab assembly is disposed along the axial direction between the first and second portions, and the second protrusion is disposed along the axial direction so as to restrain axial motion of the respective commissure tab assembly in an upstream direction.

Clause 14. The prosthetic heart valve of any of clauses 12-13, wherein, for each commissure support post, the second portion is between the first portion and an end portion of the commissure support post closest to the inflow end of the annular frame.

Clause 15. The prosthetic heart valve of any of clauses 12-14, wherein, for each commissure support post: the second protrusion includes at least one aperture, and the tabs of the corresponding commissure tab assembly are coupled to the commissure support post via one or more sutures that pass through the at least one aperture of the second protrusion.

Clause 16. The prosthetic heart valve of clause 15, wherein, for each second protrusion, each aperture is a hole or opening that extends along the axial direction through a thickness of the respective second protrusion.

Clause 17. The prosthetic heart valve of any of clauses 10-16, wherein, for each commissure support post: the first protrusion includes at least one aperture, and the tabs of the corresponding commissure tab assembly are coupled to the commissure support post via one or more sutures that pass through the at least one aperture of the first protrusion.

Clause 18. The prosthetic heart valve of clause 17, wherein, for each first protrusion, each aperture is a hole or opening that extends along the axial direction through a thickness of the respective first protrusion.

Clause 19. A prosthetic heart valve, comprising:

an annular frame that is radially collapsible and expandable between a radially-compressed configuration and a radially-expanded configuration, the annular frame having an inflow end and an outflow end separated from the inflow end along an axial direction of the frame, the annular frame comprising a plurality of commissure support posts; and

a valvular structure supported within the annular frame and comprising a plurality of leaflets, each leaflet having a pair of tabs, the tabs of adjacent leaflets being paired together to form a commissure tab assembly,

wherein there is one commissure support post for each commissure tab assembly,

each commissure tab assembly is coupled to the corresponding commissure support post with one tab wrapped at least partially around the commissure support post in a first direction and the other tab wrapped at least partially around the commissure support post in a second direction,

each commissure support post has a first portion with a pair of first protrusions that project in opposite directions along a circumferential direction of the annular frame, each first protrusion having a neck portion with a first width and an end portion with a second width greater than the first width, the neck portion being disposed along the circumferential direction between the end portion and a central portion of the first portion of the respective commissure support post such that at least one slot is formed between a surface of the central portion and a facing surface of the end portion,

for each commissure support post, the corresponding commissure tab assembly is disposed along the axial direction between the first portion and the inflow end of the annular frame, and

for each commissure support post, the pair of first protrusions is arranged along the axial direction so as to restrain axial motion of the respective commissure tab assembly in a downstream direction.

Clause 20. The prosthetic heart valve of clause 19, wherein, for each commissure support post, the first portion is an end portion thereof closest to the outflow end of the annular frame.

Clause 21. The prosthetic heart valve of any of clauses 19-20, wherein each commissure post has a second portion with a pair of second protrusions that project in opposite directions along the circumferential direction of the annular frame, each second protrusion having a second neck portion with a third width and a second end portion with a fourth width greater than the third width, the second neck portion being disposed along the circumferential direction between the second end portion and a second central portion of the second portion of the respective commissure support post such that at least one second slot is formed between a surface of the second central portion and a facing surface of the second end portion.

Clause 22. The prosthetic heart valve of clause 21, wherein, for each commissure support post: the corresponding commissure tab assembly is disposed along the axial direction between the first and second portions, and the pair of second protrusions are disposed along the axial direction so as to restrain axial motion of the respective commissure tab assembly in an upstream direction.

Clause 23. The prosthetic heart valve of any of clauses 21-22, wherein, for each commissure support post, the second portion is between the first portion and an end portion of the commissure support post closest to the inflow end of the annular frame.

Clause 24. The prosthetic heart valve of any of clauses 21-23, wherein, for each commissure support post: the tabs of the corresponding commissure tab assembly are coupled to the commissure support post via one or more sutures that pass through the second slots formed by the pair of second protrusions.

Clause 25. The prosthetic heart valve of any of clauses 21-24, wherein, for each second protrusion, the second neck portion is disposed with respect to the corresponding second end portion such that a pair of the second slots are formed on opposite sides of the second neck portion.

Clause 26. The prosthetic heart valve of any of clauses 19-25, wherein, for each commissure support post: the tabs of the corresponding commissure tab assembly are coupled to the commissure support post via one or more sutures that pass through the slots formed by the pair of first protrusions.

Clause 27. The prosthetic heart valve of any of clauses 19-26, wherein, for each first protrusion, the neck portion is disposed with respect to the corresponding end portion such that a pair of the slots are formed on opposite sides of the neck portion.

Clause 28. A prosthetic heart valve, comprising:

an annular frame that is radially collapsible and expandable between a radially-compressed configuration and a radially-expanded configuration, the annular frame having an inflow end and an outflow end separated from the inflow end along an axial direction of the frame, the annular frame comprising a plurality of commissure support posts; and

a valvular structure supported within the annular frame and comprising a plurality of leaflets, each leaflet having a pair of tabs, the tabs of adjacent leaflets being paired together to form a commissure tab assembly,

wherein there is one commissure support post for each commissure tab assembly,

each commissure tab assembly is coupled to the corresponding commissure support post with one tab wrapped at least partially around the commissure support post in a first direction and the other tab wrapped at least partially around the commissure support post in a second direction,

each commissure support post has a first portion with a first protrusion that projects along a radial direction of the annular frame toward an interior of the annular frame, each first protrusion having a neck portion with a first width and an end portion with a second width greater than the first width, the neck portion being disposed along the radial direction between the end portion and a central portion of the first portion of the respective commissure support post such that at least one slot is formed between a surface of the central portion and a facing surface of the end portion,

for each commissure support post, the corresponding commissure tab assembly is disposed along the axial direction between the first portion and the inflow end of the annular frame, and

for each commissure support post, the first protrusion is arranged along the axial direction so as to restrain axial motion of the respective commissure tab assembly in a downstream direction.

Clause 29. The prosthetic heart valve of clause 28, wherein, for each commissure support post, the first portion is an end portion thereof closest to the outflow end of the annular frame.

Clause 30. The prosthetic heart valve of any of clauses 28-29, wherein each commissure post has a second portion with a second protrusion that project along the radial direction toward the interior of the annular frame, each second protrusion having a second neck portion with a third width and a second end portion with a fourth width greater than the third width, the second neck portion being disposed along the radial direction between the second end portion and a second central portion of the second portion of the respective commissure support post such that at least one second slot is formed between a surface of the second central portion and a facing surface of the second end portion.

Clause 31. The prosthetic heart valve of clause 30, wherein, for each commissure support post: the corresponding commissure tab assembly is disposed along the axial direction between the first and second portions, and the second protrusion is arranged along the axial direction so as to restrain axial motion of the respective commissure tab assembly in an upstream direction.

Clause 32. The prosthetic heart valve of any of clauses 30-31, wherein, for each commissure support post, the second portion is between the first portion and an end portion of the commissure support post closest to the inflow end of the annular frame.

Clause 33. The prosthetic heart valve of any of clauses 30-32, wherein, for each commissure support post: the tabs of the corresponding commissure tab assembly are coupled to the commissure support post via one or more sutures that pass through the at least one second slot formed by the second protrusion.

Clause 34. The prosthetic heart valve of any of clauses 30-33, wherein, for each second protrusion, the second neck portion is disposed with respect to the corresponding second end portion such that a pair of the second slots are formed on opposite sides of the second neck portion.

Clause 35. The prosthetic heart valve of any of clauses 28-34, wherein, for each commissure support post: the tabs of the corresponding commissure tab assembly are coupled to the commissure support post via one or more sutures that pass through the at least one slot formed by the first protrusion.

Clause 36. The prosthetic heart valve of any of clauses 28-35, wherein, for each first protrusion, the neck portion is disposed with respect to the corresponding end portion such that a pair of the slots are formed on opposite sides of the neck portion.

Clause 37. A prosthetic heart valve, comprising:

an annular frame that is radially collapsible and expandable between a radially-compressed configuration and a radially-expanded configuration, the annular frame having an inflow end and an outflow end separated from the inflow end along an axial direction of the frame, the annular frame comprising a plurality of commissure support posts; and

a valvular structure supported within the annular frame and comprising a plurality of leaflets, each leaflet having a pair of tabs, the tabs of adjacent leaflets being paired together to form a commissure tab assembly, each commissure tab assembly being coupled to a respective commissure support post,

wherein the annular frame comprises means for restraining axial motion of the commissure tab assemblies.

Clause 38. The prosthetic heart valve of clause 37, wherein each commissure tab assembly is coupled to the corresponding commissure support post with one tab wrapped at least partially around the commissure support post in a first direction and the other tab wrapped at least partially around the commissure support post in a second direction.

Clause 39. The prosthetic heart valve of any one of clauses 37-38, wherein the means for restraining axial motion is constructed and arranged to restrain axial motion of the commissure tab assemblies in a downstream direction, in an upstream direction, or both.

Clause 40. The prosthetic heart valve of any one of clauses 37-39, wherein the means for restraining axial motion comprises one or more protrusions that project from respective central portions of the commissure support posts along a circumferential direction or a radial direction of the annular frame.

Clause 41. The prosthetic heart valve of any one of clauses 1-40, wherein the commissure support posts are part of an actuator assembly that actuates the annular frame between the radially-compressed configuration and the radially-expanded configuration.

Clause 42. The prosthetic heart valve of any one of clauses 1-41, wherein: each commissure support post has a first surface that faces an interior of the annular frame, a second surface that faces an exterior of the annular frame, and opposing side surfaces that extend between the first and second surfaces, and portions of the commissure tab assembly are wrapped around at least the first surface and the side surfaces of the respective commissure support post.

Clause 43. The prosthetic heart valve of any one of clauses 1-42, wherein the valvular structure is a bicuspid structure with two leaflets and two commissure tab assemblies, and the valvular structure is coupled to the annular frame via the commissure tab assemblies on diametrically opposite sides of the annular frame from each other.

Clause 44. The prosthetic heart valve of any one of clauses 1-42, wherein the valvular structure is a tricuspid structure with three leaflets and three commissure tab assemblies, and the valvular structure is coupled to the annular frame via the three commissure tab assemblies equally spaced along the circumferential direction of the annular frame.

Clause 45. The prosthetic heart valve of any one of clauses 1-44, wherein the annular frame is formed of a plastically-expandable material or a self-expanding material.

Clause 46. The prosthetic heart valve of any one of clauses 1-45, wherein the annular frame comprises an array of angled struts connected together by one or more pivot joints.

Clause 47. The prosthetic heart valve of any one of clauses 1-46, wherein the prosthetic heart valve is constructed for implantation in an existing heart valve within a patient.

Clause 48. The prosthetic heart valve of any one of clauses 1-47, wherein the prosthetic heart valve is constructed for implantation at an aortic position or a mitral position.

Clause 49. An assembly, comprising:

a delivery apparatus comprising an elongated shaft; and

the prosthetic heart valve of any one of clauses 1-48 mounted on the elongated shaft in the radially-compressed configuration for delivery into a patient's body.

Clause 50. A method of implanting a prosthetic heart valve in a patient's body, the method comprising:

inserting a distal end of a delivery apparatus into vasculature of a patient, the delivery apparatus comprising an elongated shaft, the prosthetic heart valve of any one of clauses 1-48 being releasably mounted in the radially-compressed configuration on the elongated shaft of the delivery apparatus;

advancing the prosthetic heart valve to a desired implantation site; and

using the delivery apparatus to expand the prosthetic heart valve to the radially-expanded configuration, thereby implanting the prosthetic heart valve at the desired implantation site.

Clause 51. A method of implanting a prosthetic heart valve in a patient's body, the method comprising:

inserting a distal end of a delivery apparatus into vasculature of a patient, the delivery apparatus comprising an elongated shaft, the prosthetic heart valve of any one of clauses 1-48 being releasably mounted in the radially-compressed configuration on the elongated shaft of the delivery apparatus;

advancing the prosthetic heart valve to a desired implantation site; and

deploying the prosthetic heart valve from the delivery apparatus such that the prosthetic heart valve self-expands to the radially-expanded configuration, thereby implanting the prosthetic heart valve at the desired implantation site.

Clause 52. The method of any one of clauses 50-51, wherein the advancing to the desired implantation site employs transfemoral, transventricular, transapical, or transseptal approaches.

Clause 53. A method of assembling a prosthetic heart valve, comprising:

disposing commissure tab assemblies of leaflets of a valvular structure on respective commissure support posts of an annular frame, the annular frame being radially collapsible and expandable between a radially-compressed configuration and a radially-expanded configuration, the annular frame having an inflow end and an outflow end separated from the inflow end along an axial direction of the frame, the annular frame comprising a plurality of the commissure support posts, there being one commissure support post for each commissure tab assembly, the valvular structure comprising a plurality of leaflets, each leaflet having a pair of tabs, each commissure tab assembly being formed by paired tabs of adjacent leaflets; and

coupling each commissure tab assembly to its respective commissure support post,

wherein each commissure support post has a first portion with a pair of first protrusions that project in opposite directions along a circumferential direction of the annular frame,

wherein for each commissure support post, the corresponding commissure tab assembly is disposed along the axial direction between the first portion and the inflow end of the annular frame,

wherein for each commissure support post, the pair of first protrusions is arranged along the axial direction so as to restrain axial motion of the respective commissure tab assembly in a downstream direction, and

wherein the disposing comprises:
wrapping one leaflet tab of the commissure tab assembly at least partially around the respective commissure support post in a first direction; and
wrapping another leaflet tab of the commissure tab assembly at least partially around the respective commissure support post in a second direction.

Clause 54. The method of clause 53, wherein, for each commissure support post, the first portion is an end portion thereof closest to the outflow end of the annular frame.

Clause 55. The method of any of clauses 53-54, wherein each commissure post has a second portion with a pair of second protrusions that project in opposite directions along the circumferential direction of the annular frame.

Clause 56. The method of clause 55, wherein, the disposing the commissure tab assemblies on respective commissure posts comprises: for each commissure support post, disposing the corresponding commissure tab assembly along the axial direction between the first and second portions, and for each commissure support post, the pair of second protrusions is arranged along the axial direction so as to restrain axial motion of the respective commissure tab assembly in an upstream direction.

Clause 57. The method of any of clauses 55-56, wherein, for each commissure support post, the second portion is between the first portion and an end portion of the commissure support post closest to the inflow end of the annular frame.

Clause 58. The method of any of clauses 55-57, wherein: for each commissure support post, each second protrusion includes at least one aperture, and the coupling each commissure tab assembly to its respective commissure support post comprises, for each commissure support post, passing one or more sutures through the apertures of the pair of second protrusions.

Clause 59. The method of clause 58, wherein, for each second protrusion, each aperture is a hole or opening that extends along the axial direction through a thickness of the respective second protrusion.

Clause 60. The method of any of clauses 53-59, wherein: for each commissure support post, each first protrusion includes at least one aperture, and the coupling each commissure tab assembly to its respective commissure support post comprises, for each commissure support post, passing one or more sutures through the apertures of the pair of first protrusions.

Clause 61. The method of clause 60, wherein, for each first protrusion, each aperture is a hole or opening that extends along the axial direction through a thickness of the respective first protrusion.

Clause 62. A method of assembling a prosthetic heart valve, comprising:

disposing commissure tab assemblies of leaflets of a valvular structure on respective commissure support posts of an annular frame, the annular frame being radially collapsible and expandable between a radially-compressed configuration and a radially-expanded configuration, the annular frame having an inflow end and an outflow end separated from the inflow end along an axial direction of the frame, the annular frame comprising a plurality of the commissure support posts, there being one commissure support post for each commissure tab assembly, the valvular structure comprising a plurality of leaflets, each leaflet having a pair of tabs, each commissure tab assembly being formed by paired tabs of adjacent leaflets; and

coupling each commissure tab assembly to its respective commissure support post,

wherein each commissure support post has a first portion with a first protrusion that projects along a radial direction of the annular frame toward an interior of the annular frame,

wherein for each commissure support post, the corresponding commissure tab assembly is disposed along the axial direction between the first portion and the inflow end of the annular frame,

wherein for each commissure support post, the first protrusion is arranged along the axial direction so as to restrain axial motion of the respective commissure tab assembly in a downstream direction, and

wherein the disposing comprises:
wrapping one leaflet tab of the commissure tab assembly at least partially around the respective commissure support post in a first direction; and
wrapping another leaflet tab of the commissure tab assembly at least partially around the respective commissure support post in a second direction.

Clause 63. The method of clause 62, wherein, for each commissure support post, the first portion is an end portion thereof closest to the outflow end of the annular frame.

Clause 64. The method of any of clauses 62-63, wherein each commissure post has a second portion with a second protrusion that projects along the radial direction toward the interior of the annular frame.

Clause 65. The method of clause 64, wherein, the disposing the commissure tab assemblies on respective commissure posts comprises: for each commissure support post, disposing the corresponding commissure tab assembly along the axial direction between the first and second portions, and for each commissure support post, the second protrusion is arranged along the axial direction so as to restrain axial motion of the respective commissure tab assembly in an upstream direction.

Clause 66. The method of any of clauses 64-65, wherein, for each commissure support post, the second portion is between the first portion and an end portion of the commissure support post closest to the inflow end of the annular frame.

Clause 67. The method of any of clauses 64-66, wherein: for each commissure support post, the second protrusion includes at least one aperture, and the coupling each commissure tab assembly to its respective commissure support post comprises, for each commissure support post, passing one or more sutures through at least one aperture of the second protrusion.

Clause 68. The method of clause 67, wherein, for each second protrusion, each aperture is a hole or opening that extends along the axial direction through a thickness of the respective second protrusion.

Clause 69. The method of any of clauses 62-68, wherein:

for each commissure support post, each first protrusion includes at least one aperture, and

the coupling each commissure tab assembly to its respective commissure support post comprises, for each commissure support post, passing one or more sutures through the at least one aperture of the first protrusion.

Clause 70. The method of clause 69, wherein, for each first protrusion, each aperture is a hole or opening that extends along the axial direction through a thickness of the respective first protrusion.

Clause 71. A method of assembling a prosthetic heart valve, comprising:

disposing commissure tab assemblies of leaflets of a valvular structure on respective commissure support posts of an annular frame, the annular frame being radially collapsible and expandable between a radially-compressed configuration and a radially-expanded configuration, the annular frame having an inflow end and an outflow end separated from the inflow end along an axial direction of the frame, the annular frame comprising a plurality of the commissure support posts, there being one commissure support post for each commissure tab assembly, the valvular structure comprising a plurality of leaflets, each leaflet having a pair of tabs, each commissure tab assembly being formed by paired tabs of adjacent leaflets; and

coupling each commissure tab assembly to its respective commissure support post,

wherein each commissure support post has a first portion with a pair of first protrusions that project in opposite directions along a circumferential direction of the annular frame, each first protrusion having a neck portion with a first width and an end portion with a second width greater than the first width, the neck portion being disposed along the circumferential direction between the end portion and a central portion of the first portion of the respective commissure support post such that at least one slot is formed between a surface of the central portion and a facing surface of the end portion,

wherein for each commissure support post, the corresponding commissure tab assembly is disposed along the axial direction between the first portion and the inflow end of the annular frame,

wherein for each commissure support post, the pair of first protrusions is arranged along the axial direction so as to restrain axial motion of the respective commissure tab assembly in a downstream direction, and

wherein the disposing comprises:
wrapping one leaflet tab of the commissure tab assembly at least partially around the respective commissure support post in a first direction; and
wrapping another leaflet tab of the commissure tab assembly at least partially around the respective commissure support post in a second direction.

Clause 72. The method of clause 71, wherein, for each commissure support post, the first portion is an end portion thereof closest to the outflow end of the annular frame.

Clause 73. The method of any of clauses 71-72, wherein each commissure post has a second portion with a pair of second protrusions that project in opposite directions along the circumferential direction of the annular frame, each second protrusion having a second neck portion with a third width and a second end portion with a fourth width greater than the third width, the second neck portion being disposed along the circumferential direction between the second end portion and a second central portion of the second portion of the respective commissure support post such that at least one second slot is formed between a surface of the second central portion and a facing surface of the second end portion.

Clause 74. The method of clause 73, wherein, the disposing the commissure tab assemblies on respective commissure posts comprises: for each commissure support post, disposing the corresponding commissure tab assembly along the axial direction between the first and second portions, and for each commissure support post, the pair of second protrusions is arranged along the axial direction so as to restrain axial motion of the respective commissure tab assembly in an upstream direction.

Clause 75. The method of any of clauses 73-74, wherein, for each commissure support post, the second portion is between the first portion and an end portion of the commissure support post closest to the inflow end of the annular frame.

Clause 76. The method of any of clauses 73-75, wherein the coupling each commissure tab assembly to its respective commissure support post comprises, for each commissure support post, passing one or more sutures through the second slots formed by the pair of second protrusions.

Clause 77. The method of clause 76, wherein, for each second protrusion, the second neck portion is disposed with respect to the corresponding second end portion such that a pair of the second slots are formed on opposite sides of the second neck portion.

Clause 78. The method of any of clauses 71-77, wherein the coupling each commissure tab assembly to its respective commissure support post comprises, for each commissure support post, passing one or more sutures through the slots formed by the pair of first protrusions.

Clause 79. The method of clause 78, wherein, for each first protrusion, the neck portion is disposed with respect to the corresponding end portion such that a pair of the slots are formed on opposite sides of the neck portion.

Clause 80. A method of assembling a prosthetic heart valve, comprising:

disposing commissure tab assemblies of leaflets of a valvular structure on respective commissure support posts of an annular frame, the annular frame being radially collapsible and expandable between a radially-compressed configuration and a radially-expanded configuration, the annular frame having an inflow end and an outflow end separated from the inflow end along an axial direction of the frame, the annular frame comprising a plurality of the commissure support posts, there being one commissure support post for each commissure tab assembly, the valvular structure comprising a plurality of leaflets, each leaflet having a pair of tabs, each commissure tab assembly being formed by paired tabs of adjacent leaflets; and

coupling each commissure tab assembly to its respective commissure support post,

wherein each commissure support post has a first portion with a first protrusion that projects along a radial direction of the annular frame toward an interior of the annular frame, each first protrusion having a neck portion with a first width and an end portion with a second width greater than the first width, the neck portion being disposed along the radial direction between the end portion and a central portion of the first portion of the respective commissure support post such that at least one slot is formed between a surface of the central portion and a facing surface of the end portion,

wherein for each commissure support post, the corresponding commissure tab assembly is disposed along the axial direction between the first portion and the inflow end of the annular frame,

wherein for each commissure support post, the first protrusion is arranged along the axial direction so as to restrain axial motion of the respective commissure tab assembly in a downstream direction, and

wherein the disposing comprises:
wrapping one leaflet tab of the commissure tab assembly at least partially around the respective commissure support post in a first direction; and
wrapping another leaflet tab of the commissure tab assembly at least partially around the respective commissure support post in a second direction.

Clause 81. The method of clause 80, wherein, for each commissure support post, the first portion is an end portion thereof closest to the outflow end of the annular frame.

Clause 82. The method of any of clauses 80-81, wherein each commissure post has a second portion with a second protrusion that project along the radial direction toward the interior of the annular frame, each second protrusion having a second neck portion with a third width and a second end portion with a fourth width greater than the third width, the second neck portion being disposed along the radial direction between the second end portion and a second central portion of the second portion of the respective commissure support post such that at least one second slot is formed between a surface of the second central portion and a facing surface of the second end portion.

Clause 83. The method of clause 82, wherein, the disposing the commissure tab assemblies on respective commissure posts comprises: for each commissure support post, disposing the corresponding commissure tab assembly along the axial direction between the first and second portions, and for each commissure support post, the second protrusion is arranged along the axial direction so as to restrain axial motion of the respective commissure tab assembly in an upstream direction.

Clause 84. The method of any of clauses 82-83, wherein, for each commissure support post, the second portion is between the first portion and an end portion of the commissure support post closest to the inflow end of the annular frame.

Clause 85. The method of any of clauses 82-84, wherein the coupling each commissure tab assembly to its respective commissure support post comprises, for each commissure support post, passing one or more sutures through the at least one second slot formed by the second protrusion.

Clause 86. The method of clause 85, wherein, for each second protrusion, the second neck portion is disposed with respect to the corresponding second end portion such that a pair of the second slots are formed on opposite sides of the second neck portion.

Clause 87. The method of any of clauses 80-86, wherein the coupling each commissure tab assembly to its respective commissure support post comprises, for each commissure support post, passing one or more sutures through the slots formed by the first protrusion.

Clause 88. The method of clause 87, wherein, for each first protrusion, the neck portion is disposed with respect to the corresponding end portion such that a pair of the slots are formed on opposite sides of the neck portion.

Clause 89. A method of assembling a prosthetic heart valve, comprising:

disposing commissure tab assemblies of leaflets of a valvular structure on respective commissure support posts of an annular frame, the annular frame being radially collapsible and expandable between a radially-compressed configuration and a radially-expanded configuration, the annular frame having an inflow end and an outflow end separated from the inflow end along an axial direction of the frame, the annular frame comprising a plurality of the commissure support posts, the valvular structure comprising a plurality of leaflets, each leaflet having a pair of tabs, each commissure tab assembly being formed by paired tabs of adjacent leaflets; and

coupling each commissure tab assembly to its respective commissure support post,

wherein the annular frame comprises means for restraining axial motion of the commissure tab assemblies.

Clause 90. The method of clause 89, wherein the disposing the commissure tab assemblies on the respective commissure support posts comprises: wrapping one leaflet tab of the commissure tab assembly at least partially around the respective commissure support post in a first direction; and wrapping another leaflet tab of the commissure tab assembly at least partially around the respective commissure support post in a second direction.

Clause 91. The method of any one of clauses 89-90, wherein the means for restraining axial motion is constructed and arranged to restrain axial motion of the commissure tab assemblies in a downstream direction, in an upstream direction, or both.

Clause 92. The method of any one of clauses 89-91, wherein the means for restraining axial motion comprises one or more protrusions that project from respective central portions of the commissure support posts along a circumferential direction or a radial direction of the annular frame.

Clause 93. The method of any one of clauses 53-92, wherein the commissure support posts are part of an actuator assembly that actuates the annular frame between the radially-compressed configuration and the radially-expanded configuration.

Clause 94. The method of any one of clauses 53-93, wherein: each commissure support post has a first surface that faces an interior of the annular frame, a second surface that faces an exterior of the annular frame, and opposing side surfaces that extend between the first and second surfaces, and the disposing the commissure tab assemblies on the respective commissure support posts is such that portions of the commissure tab assembly are wrapped around at least the first surface and the side surfaces of the respective commissure support post.

Clause 95. The method of any one of clauses 53-94, wherein the valvular structure is a bicuspid structure with two leaflets and two commissure tab assemblies, and the commissure support posts are on diametrically opposite sides of the annular frame from each other.

Clause 96. The method of any one of clauses 53-94, wherein the valvular structure is a tricuspid structure with three leaflets and three commissure tab assemblies, and the commissure support posts are equally spaced around a circumference of the annular frame.

Clause 97. The method of any one of clauses 53-96, wherein the annular frame is formed of a plastically-expandable material or a self-expanding material.

Clause 98. The method of any one of clauses 53-97, wherein the annular frame comprises an array of angled struts connected together by one or more pivot joints.

Clause 100. A prosthetic heart valve, comprising:

an expandable annular frame having a longitudinal axis and comprising a plurality of commissure support posts; and

a valvular structure supported by the frame and comprising a plurality of leaflets, each leaflet having a pair of tabs, the tabs of adjacent leaflets of the leaflet assembly being paired together to form a commissure tab assembly,

wherein there is one commissure support post for each commissure tab assembly,

wherein each commissure tab assembly is coupled to the respective commissure support post with one tab wrapped at least partially around the commissure support post in a first direction and the other tab wrapped at least partially around the commissure support post in a second direction, and

wherein at least a first portion of each commissure support post includes at least one first protrusion that extends in a direction perpendicular to the longitudinal axis and is constructed to restrain axial motion of the respective commissure tab assembly in a downstream direction.

Clause 101. The prosthetic heart valve of clause 100, wherein, for each commissure support post, the first portion is an outflow end portion thereof.

Clause 102. The prosthetic heart valve of any of clauses 100-101, wherein one or more of the first protrusions projects radially toward an interior of the annular frame.

Clause 103. The prosthetic heart valve of any one of clause 100-102, wherein one or more of the first protrusions projects along a circumferential direction of the annular frame.

Clause 104. The prosthetic heart valve of clause 103, wherein: one or more of the first protrusions has a first portion with a first width, and a second portion with a second width greater than the first width, the first portion being closer to a center of the respective commissure support post than the second portion such that at least one slot is formed between a surface of the respective commissure support post and a facing surface of the second portion, and the commissure tab assembly is coupled to the respective commissure support post via a suture that passes through a portion of the commissure tab assembly and through the at least one slot.

Clause 105. The prosthetic heart valve of any one of clauses 103-104, wherein one or more of the commissure support posts includes a pair of said first protrusions that project along the circumferential direction, one of the pair being on an opposite side of the commissure support post along the circumferential direction from the other of the pair.

Clause 106. The prosthetic heart valve of any of clauses 100-105, wherein: one or more of the first protrusions includes at least one aperture, and the commissure tab assembly is coupled to the respective commissure support post via a suture that passes through one or more of the tabs and through the at least one aperture of the first protrusion.

Clause 107. The prosthetic heart valve of any of clauses 100-106, wherein a second portion of one or more of the commissure support posts includes at least one second protrusion that extends in a direction perpendicular to the longitudinal axis and is constructed to restrain movement of the respective commissure tab assembly in an upstream direction.

Clause 108. The prosthetic heart valve of clause 107, wherein one or more of the second protrusions projects radially toward an interior of the annular frame.

Clause 109. The prosthetic heart valve of any one of clauses 107-108, wherein one or more of the second protrusions projects along a circumferential direction of the annular frame.

Clause 110. The prosthetic heart valve of clause 109, wherein: one or more of the second protrusions has a first portion with a first width, and a second portion with a second width greater than the first width, the first portion being closer to a center of the respective commissure support post than the second portion such that at least one slot is formed between a surface of the respective commissure support post and a facing surface of the second portion, and the commissure tab assembly is coupled to the respective commissure support post via a suture that passes through a portion of the commissure tab assembly and through the at least one slot.

Clause 111. The prosthetic heart valve of any one of clauses 109-110, wherein one or more of the commissure support posts includes a pair of said second protrusions that project along the circumferential direction, one of the pair being on an opposite side of the commissure support post along the circumferential direction from the other of the pair.

Clause 112. The prosthetic heart valve of any one of clauses 107-111, wherein: one or more of the second protrusions includes at least one aperture, and each commissure tab assembly is coupled to the respective commissure support post via a suture that passes through the tabs and through the at least one aperture of the second protrusion.

Clause 113. The prosthetic heart valve of any one of clauses 111-112, wherein the commissure support posts are part of an actuator assembly that actuates the annular frame from an insertable, unexpanded state to an installed, expanded state.

Clause 114. The prosthetic heart valve of any one of clauses 100-113, wherein: each commissure support post has a first surface that faces an interior of the annular frame, a second surface that faces an exterior of the annular frame, and opposing side surfaces that extend between the first and second surfaces, and portions of the commissure tab assembly are wrapped around at least the first and side surfaces of the respective commissure support post.

Clause 115. The prosthetic heart valve of any one of clauses 100-114, wherein the first direction is opposite to the second direction.

Clause 116. A method of assembling a prosthetic heart valve, comprising:

disposing commissure tab assemblies of a valvular structure on respective commissure support posts of an expandable annular frame having a longitudinal axis, each commissure support post having a first portion and a second portion, the first portion being closer to an outflow end of the annular frame than the second portion; and

coupling each commissure tab assembly to the respective commissure support post,

wherein the disposing includes:
wrapping one leaflet tab of the commissure tab assembly at least partially around the respective commissure support post in a first direction; and
wrapping another leaflet tab of the commissure tab assembly at least partially around the respective commissure support post in a second direction, and

wherein at least the first portion of each commissure support post includes at least one first protrusion that extends in a direction perpendicular to the longitudinal axis and is constructed to restrain movement of the respective commissure tab assembly toward said outflow end of the annular frame.

Clause 117. The method of clause 116, wherein the first direction is opposite to the second direction.

Clause 118. The method of any one of clauses 116-117, comprising: prior to the disposing, coupling together a plurality of leaflets at adjacent tabs thereof to form the leaflet assembly, the coupled tabs forming the respective commissure tab assemblies.

Clause 119. The method of any one of clauses 116-118, wherein one or more of the first protrusions projects radially toward an interior of the annular frame.

Clause 120. The method of any one of clauses 116-119, wherein one or more of the first protrusions projects along a circumferential direction of the annular frame.

Clause 121. The method of clause 120, wherein: one or more of the first protrusions has a first portion with a first width, and a second portion with a second width greater than the first width, the first portion being closer to a center of the respective commissure support post than the second portion such that at least one slot is formed between a surface of the respective commissure support post and a facing surface of the second portion, and the coupling the commissure tab assembly comprises passing a suture through one or more of the leaflet tabs and through the at least one slot formed by the first protrusion of the respective commissure support post.

Clause 122. The method of any one of clauses 120-121, wherein one or more of the commissure support posts includes a pair of said first protrusions that project along the circumferential direction, one of the pair being on an opposite side of the commissure support post along the circumferential direction from the other of the pair.

Clause 123. The method of any one of clauses 116-122, wherein the coupling each commissure tab assembly comprises attaching the commissure tab assembly to the at least one first protrusion of the respective commissure post via a suture.

Clause 124. The method of clause 123, wherein one or more of the first protrusions includes at least one aperture, and the coupling the commissure tab assembly comprises passing the suture through one or more of the leaflet tabs and through the at least one aperture of the first protrusion.

Clause 125. The method of any one of clauses 116-124, wherein the second portion of one or more of the commissure support posts includes at least one second protrusion that extends in a direction perpendicular to the longitudinal axis and is constructed to restrain movement of the respective commissure tab assembly away from said axial end of the annular frame.

Clause 126. The method of clause 125, wherein the disposing comprises positioning each commissure tab assembly between the first and second protrusions of the respective commissure support post along an axial direction of the annular frame.

Clause 127. The method of any one of clauses 125-126, wherein one or more of the second protrusions projects radially toward an interior of the annular frame.

Clause 128. The method of any one of clauses 125-127, wherein one or more of the second protrusions projects along a circumferential direction of the annular frame.

Clause 129. The method of clause 128, wherein one or more of the commissure support posts includes a pair of said second protrusions that project along the circumferential direction, one of the pair being on an opposite side of the commissure support post along the circumferential direction from the other of the pair.

Clause 130. The method any one of clauses 125-129, wherein the coupling each commissure tab assembly comprises attaching the commissure tab assembly to the at least one second protrusion of the respective commissure support post via a suture.

Clause 131. The method of clause 130, wherein one or more of the second protrusions includes at least one aperture, and the attaching the commissure tab assembly comprises passing the suture through one or more of the leaflet tabs and through the at least one aperture of the second protrusion.

Clause 132. The method of clause 130, wherein: one or more of the second protrusions has a first portion with a first width, and a second portion with a second width greater than the first width, the first portion being closer to a center of the respective commissure support post than the second portion such that at least one slot is formed between a surface of the respective commissure support post and a facing surface of the second portion, and the coupling the commissure tab assembly comprises passing a suture through one or more of the leaflet tabs and through the at least one slot formed by the second protrusion.

### General Considerations

All features described herein are independent of one another and, except where structurally impossible, can be used in combination with any other feature described herein. For example, the inwardly-directed protrusion 364 of FIGS. 10A-11B and/or the inwardly-directed protrusion 382 of FIGS. 11A-11B can be used in combination with any of the circumferentially-directed protrusions described with respect to FIGS. 6A-9B. In another example, the inwardly-directed protrusion 230 of FIG. 4A-5B and/or the inwardly-directed protrusion 232 of FIGS. 5A-5B can be used in combination with any of the circumferentially-directed protrusions described with respect to FIGS. 6A-9B.

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present, or problems be solved. The technologies from any example can be combined with the technologies described in any one or more of the other examples.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used herein with reference to the prosthetic heart valve assembly and implantation and structures of the prosthetic heart valve, “proximal” refers to a position, direction, or portion of a component that is closer to the user and a handle of the delivery system or apparatus that is outside the patient, while “distal” refers to a position, direction, or portion of a component that is further away from the user and the handle, and closer to the implantation site. The terms “longitudinal” and “axial” refer to an axis extending in the proximal and distal directions, unless otherwise expressly defined.

The terms “axial direction,” “radial direction,” and “circumferential direction” have been used herein to describe the arrangement and assembly of components relative to the geometry of the frame of the prosthetic heart valve. Such terms have been used for convenient description, but the disclosed embodiments are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language. As used herein, “and/or” means “and” or “or,” as well as “and” and or.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” “right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

In view of the many possible embodiments to which the principles of the disclosed technology may be applied, it should be recognized that the illustrated examples are only preferred examples and should not be taken as limiting the scope of the disclosed technology. Rather, the scope of the invention is defined by the following claims. We therefore claim as our invention all that comes within the scope and spirit of these claims.

1. A prosthetic heart valve comprising:
an annular frame that is radially collapsible and expandable between a radially-compressed configuration and a radially-expanded configuration, the annular frame having an inflow end and an outflow end separated from the inflow end along an axial direction of the frame, the annular frame comprising a first commissure support post comprising a first protrusion and a second protrusion; and
a valvular structure supported within the annular frame and comprising a first leaflet, a second leaflet, and a commissure tab assembly, each of the first leaflet and the second leaflet comprising a pair of tabs, wherein adjacent tabs of the first leaflet and the second leaflet forms a first commissure tab assembly,
wherein the first commissure tab assembly is coupled to the first support post;
wherein the first commissure tab assembly is disposed along the axial direction between the first protrusion and the inflow end of the annular frame; and
wherein the set of first protrusions is configured to limit axial movement of the first commissure tab assembly in a downstream direction.

2. The prosthetic heart valve of claim 1, wherein each of the first protrusion and the second protrusion comprises:
a neck portion with a first width; and
an end portion with a second width greater than the first width,
wherein the neck portion is disposed along the circumferential direction between the end portion and the first commissure support post such that at least one slot is formed between the first commissure support port and the end portion.

3. The prosthetic heart valve of claim 2, wherein the tabs of the first commissure tab assembly are coupled to the first commissure support post via one or more sutures that pass through the at least one slot.

4. The prosthetic heart valve of claim 1, wherein each of the first protrusion and the second protrusion comprises at least one aperture, and wherein the tabs of the first commissure tab assembly are coupled to the first commissure support post via one or more sutures that pass through the at least one aperture.

5. The prosthetic heart valve of claim 1, wherein the first protrusion and the second protrusion project in opposite circumferential directions of the annular frame.

6. The prosthetic heart valve of claim 5, further comprising a third protrusion and a fourth protrusion, wherein the third protrusion and the fourth protrusion are positioned axially apart from the first protrusion and the second protrusion, and wherein the third protrusion and the fourth protrusion project in opposite circumferential directions of the annular frame.

7. The prosthetic heart valve of claim 1, wherein the second protrusion is positioned axially apart from the first protrusion, and wherein the first protrusion and the second protrusion project along a radial direction toward the interior of the annular frame.

8. The prosthetic heart valve of claim 7, wherein the first commissure tab assembly is positioned between the first protrusion and the second protrusion.

9. The prosthetic heart valve of claim 7, wherein the second protrusion is configured to limit axial movement of the first commissure tab assembly in an upstream direction.

10. The prosthetic heart valve of claim 1, wherein the first commissure support post is a part of an actuator assembly configured to actuate the annular frame between the radially-compressed configuration and the radially-expanded configuration.

11. A prosthetic heart valve comprising:
an annular frame that is radially collapsible and expandable between a radially-compressed configuration and a radially-expanded configuration, the annular frame having an inflow end and an outflow end separated from the inflow end along an axial direction of the frame, the annular frame comprising a first commissure support post comprising a first protrusion; and
a valvular structure supported within the annular frame and comprising a first leaflet, a second leaflet, and a commissure tab assembly, each of the first leaflet and the second leaflet comprising a pair of tabs, wherein adjacent tabs of the first leaflet and the second leaflet forms a first commissure tab assembly,
wherein the first commissure tab assembly is coupled to the first support post;
wherein the first commissure tab assembly is disposed along the axial direction between the first protrusion and the inflow end of the annular frame; and
wherein the first protrusion projects along a radial direction toward the interior of the annular frame.

12. The prosthetic heart valve of claim 11, wherein the first protrusion includes at least one aperture, and wherein the tabs of the first commissure tab assembly are coupled to the first commissure support post via one or more sutures that pass through the at least one aperture of the set of first protrusions.

13. The prosthetic heart valve of claim 11, wherein the first protrusion comprises:
a neck portion with a first width; and
an end portion with a second width greater than the first width,
wherein the neck portion is disposed along the circumferential direction between the end portion and the first commissure support post such that at least one slot is formed between the first commissure support port and the end portion.

14. The prosthetic heart valve of claim 13, wherein the tabs of the first commissure tab assembly are coupled to the first commissure support post via one or more sutures that pass through the at least one slot associated with the first protrusion.

15. The prosthetic heart valve of claim 16, wherein the first protrusion is configured to limit axial movement of the first commissure tab assembly in a downstream direction.

16. The prosthetic heart valve of claim 16, wherein the first commissure support post is a part of an actuator assembly configured to actuate the annular frame between the radially-compressed configuration and the radially-expanded configuration.

17. A method of assembling a prosthetic heart valve, comprising:
providing an annular frame comprising a first commissure support post, the annular frame being radially collapsible and expandable between a radially-compressed configuration and a radially-expanded configuration, the annular frame having an inflow end and an outflow end separated from the inflow end along an axial direction of the annular frame, the first commissure port comprising a pair of first protrusions projecting in opposite directions along a circumferential direction of the annular frame;
providing a first commissure tab assembly of leaflets of a valvular structure, the first commissure assembly comprising tabs of adjacent leaflets;
coupling the first commissure tab assembly to the first commissure support post,
wherein the first commissure tab assembly is disposed along the axial direction of the annular frame between the first portion and the inflow end of the annular frame,
wherein the pair of first protrusions is configured to restrain axial motion of the first commissure tab assembly in a downstream direction, and
wherein the coupling the first commissure tab assembly to the first commissure support post comprises:
wrapping a first leaflet tab of the first commissure tab assembly at least partially around the first commissure support post in a first direction; and
wrapping a second leaflet tab of the first commissure tab assembly at least partially around the first commissure support post in a second direction.

18. The method of claim 17, wherein the first commissure post comprises a pair of second protrusions that project in opposite directions along the circumferential direction of the annular frame, and wherein the coupling the first commissure tab assembly to the first commissure support post comprises:
disposing the first commissure tab assembly along the axial direction between the pair of first protrusions and the pair of second protrusions,
wherein the pair of second protrusions is configured to restrain axial motion of the respective commissure tab assembly in an upstream direction.

19. The method of claim 17, wherein the pair of first protrusions comprises at least one aperture, and wherein the coupling the first commissure tab assembly to the first commissure support post comprises passing one or more sutures through the at least one apertures of the pair of first protrusions.

20. The method of claim 17, wherein each first protrusion of the pair of first protrusions comprises:
a neck portion with a first width; and
an end portion with a second width greater than the first width,
wherein the neck portion is disposed along the circumferential direction between the end portion and the first commissure support post such that at least one slot is formed between the first commissure support port and the end portion; and
wherein the coupling the first commissure tab assembly to the first commissure support post comprises passing one or more sutures through the at least one slot of the pair of first protrusions.
